(19) United States
(12) Patent Application Publication
(10) Pub. No.: US 2025/0146973 A1
(43) Pub. Date: May 8, 2025

(54) LABEL-FREE METHODS OF SENSING

(71) Applicant: Board of Regents, The University of Texas System, Austin, TX (US)

(72) Inventors: Georgios ALEXANDRAKIS, Arlington, TX (US); Scott RENKES, Arlington, TX (US)

(21) Appl. No.: 18/730,176

(22) PCT Filed: Jan. 20, 2023
(86) PCT No.: PCT/US2023/011259

§ 371 Date: Jul. 18, 2024

Related U.S. Application Data
(60) Provisional application No. 63/301,614, filed on Jan. 21, 2022.

(51) Int. Cl. G01N 27/447; G01N 33/543; G01N 33/569

(52) U.S. Cl. CPC G01N 27/44791; G01N 27/4473; G01N 33/5438; G01N 33/56977

(57) ABSTRACT

Methods of sensing are disclosed. In some embodiments, a method comprises providing a sensor. The sensor comprises a first layer having a nanohole structure, and a second layer having a nanopore. The nanohole structure is aligned with the nanopore in a translocation direction. The method further comprises contacting a test sample comprising an analyte with the first layer of the sensor. The nanohole structure is irradiated with light, and the analyte is optically trapped in the nanohole structure. The method further comprises applying a first electric field (DC) across the nanopore to draw analyte into the nanopore, and then applying a second electric field (pulsed, modulated or AC) across the nanopore. The method further comprises measuring a change in current and/or phase across the nanopore during application of the second electric field or measuring at least one kinetic parameter of the analyte within the nanopore after removing the second field.

### CROSS-REFERENCE TO RELATED APPLICATIONS

This application claims priority to U.S. Provisional Patent Application No. 63/301,614 filed on Jan. 21, 2022, the entire contents of which are incorporated herein by reference.

### STATEMENT REGARDING FEDERALLY SPONSORED RESEARCH

This invention was made with government support under Grant No. 1R21CA240220-01A1 awarded by the National Cancer Institute and Grant No. T32 HL134613 awarded by the National Heart, Lung, and Blood Institute. The Government has certain rights in the invention.

### FIELD

The present application relates to methods of sensing, including label-free methods of sensing.

### BACKGROUND

Nanopores can be used to discriminate between analytes through the analysis of changes in conduction current profiles during translocation. However, the translocation times of analytes through some nanopore-based sensors are extremely fast, which limits the fidelity of electrical data that can be collected. Additionally, some nanopore-based sensing methods provide only certain types of data regarding analytes. There is a need for improved methods of sensing using nanopores, including methods that provide additional data regarding analytes and that can differentiate between additional types of analytes.

### SUMMARY

In one aspect, methods of sensing are described herein. In some embodiments, such a method comprises providing a sensor. The sensor comprises a first layer having at least one single nanohole structure or at least one dual nanohole structure, and a second layer having at least one nanopore. The single nanohole structure comprises only one nanohole. The dual nanohole structure comprises a first nanohole and a second nanohole connected by a gap. Additionally, the one nanohole (in the case of the single nanohole structure) or the gap (in the case of a dual nanohole structure) of the first layer is aligned with the nanopore of the second layer in a direction corresponding to a translocation direction across the first and second layers.

Sensors described herein having a dual nanohole structure can have any construction, structure, or property of a dual nanohole sensor described hereinbelow. Similarly, a sensor described herein having a single nanohole structure (rather than a dual nanohole structure) can likewise have any construction, structure, or property of a sensor described below.

Turning again to the method steps, a method described herein further comprises providing a test sample comprising an analyte and contacting the test sample with the first layer of the sensor. The method also comprises irradiating the single nanohole structure or the dual nanohole structure of the sensor with a beam of electromagnetic radiation and optically trapping the analyte in the single nanohole structure or in the dual nanohole structure and/or in the gap of the first layer of the sensor.

Moreover, in some cases, the method further comprises applying a first electric field across the nanopore to draw one or more of the analytes into the nanopore, wherein the first electric field comprises a direct current (DC) electric field. The method also comprises applying a second electric field across the nanopore after applying the first electric field, wherein the second electric field comprises a pulsed, modulated, or alternating current (AC) electric field.

In addition, in some implementations, a method described herein further comprises measuring one or more analyte properties or other properties potentially associated with optical trapping or translocation of the analyte through the nanopore of the sensor. For example, in some cases, a method described herein further comprises measuring a change in current and/or phase across the nanopore during application of the second electric field while the analyte is optically trapped and/or during one or more translocation events of the analyte through the nanopore. A method described herein can also (or alternatively) comprise measuring at least one kinetic parameter of the analyte within the nanopore after removing or turning off the second electric field. Further, in some embodiments, at least one kinetic parameter is measured while the analyte decelerates or comes to a stop while optically trapped.

In addition, in some cases, measuring change in current and/or phase further comprises determining a charge of a translocating analyte. Moreover, in some instances, measuring change in current and/or phase further comprises determining a dielectric constant of a translocating analyte.

Methods of sensing described herein, in some implementations, further comprise measuring a surface plasmon resonance of the single nanohole structure or the dual nanohole structure after optically trapping the analyte in the single nanohole structure or in the dual nanohole structure and/or in the gap of the first layer of the sensor. Additionally, in some such embodiments, measuring the surface plasmon resonance further comprises determining the mass of the optically trapped analyte.

Methods described herein can be used for sensing a variety of analytes. For example, in some cases, the analyte comprises complexed and/or non-complexed biomolecules. In other instances, the analyte comprises a nanoparticle such as an inorganic nanoparticle.

Additional features and embodiments are further described in the detailed description which follows.

### BRIEF DESCRIPTION OF THE FIGURES

FIG. 1 schematically illustrates a sensor and steps of a sensing method according to one embodiment described herein.

FIG. 2A is a plot of the simultaneously recorded Ipatch current response (pA, top panel) and command voltage (mV, bottom panel) versus time(s) from a 100 Hz AC oscillation taken during a 70 pM Au nanoparticle run. The phase shift and post-drive decay (20) are shown.

FIG. 2B is an example plot of the 70 pM Au nanoparticle post-drive decay (pA) versus time(s) highlighted in FIG. 2A. The damped oscillation was fit to the post-drive decay that was trimmed at the termination of the command voltage oscillation, and the fit is also plotted.

FIG. 3A is a plot of the phase shift (deg) versus the frequency (Hz) of the empty trap and model cell response. The plot along the axis of the right side of the graph displays the delta phase shift (deg), which is plotted as a solid line.

FIG. 3B is a plot of the conductance (I/V) versus the frequency (Hz) of a sensor according to one embodiment described herein. The plot along the axis of the right side of the graph displays the ratio of the empty trap response to the model cell response, which is plotted as a solid line.

FIG. 4A is a plot of the delta phase shift (deg) of the phase response of 1 fM SiO₂ nanoparticles and the empty trap at 100 Hz driving frequency.

FIG. 4B is a plot of the ratio of the conductance of the 1 fM SiO₂ nanoparticle solution and the empty trap versus frequency (Hz).

FIG. 4C is a plot of the phase shift (deg) of Au and SiO₂ nanoparticle solutions compared to the empty trap versus frequency (Hz) at 100 Hz driving frequency.

FIG. 4D is a plot of the conductance (I/V) of Au and SiO₂ nanoparticle solutions compared to an empty trap versus frequency (Hz) at 100 Hz driving frequency.

FIG. 5A is a box and whisker plot for the intercept of regression parameter (a1) for the fit y=a1+a2*x+b1*sin(c1*x+d1)*e^{e1*x} for the empty sensor and the sensor tested with 1 fM and 70 pM Au nanoparticle solutions and 1 fM and 50 fM SiO₂ nanoparticle solutions, according to some embodiments described herein.

FIG. 5B is a box and whisker plot for the magnitude of oscillation for regression parameter (decay magnitude, b1) for the fit y=a1+a2*x+b1*sin(c1*x+d1)*e^{e1*x} for the empty sensor and the sensor tested with 1 fM and 70 pM Au nanoparticle solutions and 1 fM and 50 fM SiO₂ nanoparticle solutions, according to some embodiments described herein.

FIG. 5C is a box and whisker plot for the frequency response of the post-drive decay parameter (decay frequency, cl, Hz) for the fit y=a1+a2*x+b1*sin(c1*x+d1)*e^{e1*x} for the empty sensor and the sensor tested with 1 fM and 70 pM Au nanoparticle solutions and 1 fM and 50 fM SiO₂ nanoparticle solutions, according to some embodiments described herein. The oval indicates that 1 fM SiO₂ nanoparticle solutions have similar frequency as the outliers of the 50 fM SiO₂ group.

FIG. 5D is a box and whisker plot for the slope of the linear drift component parameter (a2) for the fit y=a1+a2*x+b1*sin(c1*x+d1)*e^{e1*x} for the empty sensor and the sensor tested with 1 fM and 70 pM Au nanoparticle solutions and 1 fM and 50 fM SiO₂ nanoparticle solutions, according to some embodiments described herein.

FIG. 5E is a box and whisker plot for the post-drive decay oscillation phase parameter (decay phase, d1) for the fit y=a1+a2*x+b1*sin(c/*x+d1)*e^{e1*x} for the empty sensor and the sensor tested with 1 fM and 70 pM Au nanoparticle solutions and 1 fM and 50 fM SiO₂ nanoparticle solutions, according to some embodiments described herein. The oval indicates that the 50 fM SiO₂ group correlated with the values of the 1 fM SiO₂ group.

FIG. 5F is a box and whisker plot for the decay coefficient for the damped oscillation envelope parameter (e1) for the fit y=a1+a2*x+b1*sin(c1*x+d1)*e^{e1*x} for the empty sensor and the sensor tested with 1 fM and 70 pM Au nanoparticle solutions and 1 fM and 50 fM SiO₂ nanoparticle solutions, according to some embodiments described herein.

FIG. 6A shows staining results for yeast binding to Cd-2/HLA-A2 target antigen at 100 nM after two rounds of FACS sorting, according to some embodiments described herein. The gated region in the histogram marked with a star in quadrant 2 (Q2) reveals yeast displaying antibodies that recognize the CdK-2/HLA-A2 antigen. Antibody expression on the surface of yeast was detected with an anti-FLAG tag-FITC conjugate (y-axis). Biotin-labeled pMHC antigen bound to yeast was detected with SA-PE (x-axis).

FIG. 6B shows staining results for yeast binding to Cd-2/HLA-A2 target antigen at 10 nM after two rounds of FACS sorting, according to some embodiments described herein. The gated region in the histogram marked with a star in quadrant 2 (Q2) reveals yeast displaying antibodies that recognize the CdK-2/HLA-A2 antigen. Antibody expression on the surface of yeast was detected with an anti-FLAG tag-FITC conjugate (y-axis). Biotin-labeled pMHC antigen bound to yeast was detected with SA-PE (x-axis).

FIG. 6C displays FACS sorting results for yeast binding to a negative control at 100 nM, according to some embodiments described herein. Antibody expression on the surface of yeast was detected with an anti-FLAG tag-FITC conjugate (y-axis). Biotin-labeled pMHC antigen bound to yeast was detected with SA-PE (x-axis).

FIG. 6D shows FACS sorting results for yeast binding to an additional negative control at 100 nM, according to some embodiments described herein. Antibody expression on the surface of yeast was detected with an anti-FLAG tag-FITC conjugate (y-axis). Biotin-labeled pMHC antigen bound to yeast was detected with SA-PE (x-axis).

FIG. 6E illustrates FACS sorting results for yeast binding with no antigen present, according to some embodiments described herein. Antibody expression on the surface of yeast was detected with an anti-FLAG tag-FITC conjugate (y-axis). Biotin-labeled pMHC antigen bound to yeast was detected with SA-PE (x-axis).

FIG. 7A is a histogram of the optical step change (%) and trapping event counts for RAH (pMHC), anti-RAH (TCRm), and their equimolar mixture (RAH-anti-RAH) compiled from multimodal optical-electrical sensor data, according to some embodiments described herein.

FIG. 7B is a histogram of the trapping current (nA) and trapping event counts for RAH (pMHC), anti-RAH (TCRm), and their equimolar mixture (RAH-anti-RAH) compiled from multimodal optical-electrical sensor data, according to some embodiments described herein.

FIG. 7C is a histogram of the nanopore translocation current spikes (nA) and trapping event counts for RAH (pMHC), anti-RAH (TCRm), and their equimolar mixture (RAH-anti-RAH) compiled from multimodal optical-electrical sensor data, according to some embodiments described herein.

FIG. 8 is a graph of the Lab VIEW-driven pulse train that contains 10 cycles of sequential sinusoidal frequency bursts, each of 10, 20, 50, 100, 200, 500, 1000, 2000, 5000, 10000, 20000, 50000, and 100000 Hz, according to some embodiments described herein.

FIG. 9A is a plot of the phase shift (deg) measured relative to the empty sensor for RAH (pMHC) and anti-RAH (TCRm) solutions at 1 aM, according to some embodiments described herein.

FIG. 9B is a plot of the frequency-dependent conductance (I/V) measured relative to the empty sensor for RAH (pMHC) and anti-RAH (TCRm) solutions at 1 aM, according to some embodiments described herein.

FIG. 10A is a plot of Ipatch current response (pA) versus time(s) from AC oscillations, according to some embodiments described herein.

FIG. 10B is a plot of the command voltage (mV) versus time(s) recorded simultaneously as the data acquired in FIG. 10A from AC oscillations.

FIG. 11 is a further close-up of a section of one of the frequency bursts of the command voltage (mV) plotted in FIG. 10B versus time(s).

FIG. 12A is a plot of the optical voltage (V) versus time(s) recorded simultaneously as the data acquired in FIG. 12B from an AC oscillation plot for 1 aM RAH-anti-RAH.

FIG. 12B is a plot of the command voltage (mV) versus time(s) recorded simultaneously as the data acquired in FIG. 12A from an AC oscillation plot for 1 aM RAH-anti-RAH.

FIG. 12C is a plot of the Ipatch current response (pA) versus time(s) from an AC oscillation plot for 1 aM RAH-anti-RAH.

FIG. 12D is a plot of the OpticalRe (V) versus time(s) recorded simultaneously as the data acquired in FIG. 12C from an AC oscillation plot for 1 aM RAH-anti-RAH.

FIG. 13A is an expanded view of applied voltage frequency burst like the ones shown in FIG. 12B, illustrating a plot of the command voltage (mV) versus time(s) recorded simultaneously during an AC oscillation at 1 aM RAH-anti-RAH.

FIG. 13B is an expanded view of the sensor current response when an analyte is trapped in it (an RAH-anti-RAH protein complex) in response to the externally applied command voltage shown in FIG. 13A. The circle indicates the post-drive decay shown in FIG. 14.

FIG. 14 is a plot of the 1 aM RAH-anti-RAH post-drive decay (pA) versus time(s). The damped oscillation was fit to the post-drive decay that was trimmed at the termination of the command voltage oscillation, and the fit is also plotted.

FIG. 15A is a box and whisker plot for the intercept of regression parameter (a1) for the fit y=a1+a2*x+b1*sin(c1*x+d1)*e^{e1*x} for 1 aM RAH, 1 aM anti-RAH, and 1 aM RAH-anti-RAH from a 100 kHz post-drive decay.

FIG. 15B is a box and whisker plot for the magnitude of oscillation for regression parameter (decay magnitude, b1) for the fit y=a1+a2*x+b1*sin(c1*x+d1)*e^{e1*x} for 1 aM RAH, 1 aM anti-RAH, and 1 aM RAH-anti-RAH from a 100 kHz post-drive decay.

FIG. 15C is a box and whisker plot for the frequency response of the post-drive decay parameter (decay frequency, cl, Hz) for the fit y=a1+a2*x+b1*sin(c1*x+d1)*e^{e1*x} for 1 aM RAH, 1 aM anti-RAH, and 1 aM RAH-anti-RAH from a 100 kHz post-drive decay.

FIG. 15D is a box and whisker plot for the slope of the linear drift component parameter (a2) for the fit y=a1+a2*x+b1*sin(c1*x+d1)*e^{e1*x} for 1 aM RAH, 1 aM anti-RAH, and 1 aM RAH-anti-RAH from a 100 kHz post-drive decay.

FIG. 15E is a box and whisker plot for the post-drive decay oscillation phase parameter (decay phase, d1) for the fit y=a1+a2*x+b1*sin(c1*x+d1)*e^{e1*x} for 1 aM RAH, 1 aM anti-RAH, and 1 aM RAH-anti-RAH from a 100 kHz post-drive decay.

FIG. 15F is a box and whisker plot for the decay coefficient for the damped oscillation envelope parameter (e1) for the fit y=a1+a2*x+b1*sin(c1*x+d1)*e^{e1*x} for 1 aM RAH, 1 aM anti-RAH, and 1 aM RAH-anti-RAH from a 100 kHz post-drive decay.

FIG. 16A is a box and whisker plot for the intercept of regression parameter (a1) for the fit y=a1+a2*x+b1*sin(c1*x+d1)*e^{e1*x} for 1 aM RAH, 1 aM anti-RAH, and 1 aM RAH-anti-RAH from a 1 kHz post-drive decay.

FIG. 16B is a box and whisker plot for the magnitude of oscillation for regression parameter (decay magnitude, b1) for the fit y=a1+a2*x+b1*sin(c1*x+d1)*e^{e1*x} for 1 aM RAH, 1 aM anti-RAH, and 1 aM RAH-anti-RAH from a 1 kHz post-drive decay.

FIG. 16C is a box and whisker plot for the frequency response of the post-drive decay parameter (decay frequency, cl, Hz) for the fit y=a1+a2*x+b1*sin(c1*x+d1)*e^{e1*x} for 1 aM RAH, 1 aM anti-RAH, and 1 aM RAH-anti-RAH from a 1 kHz post-drive decay.

FIG. 16D is a box and whisker plot for the slope of the linear drift component parameter (a2) for the fit y=a1+a2*x+b1*sin(c1*x+d1)*e^{e1*x} for 1 aM RAH, 1 aM anti-RAH, and 1 aM RAH-anti-RAH from a 1 kHz post-drive decay.

FIG. 16E is a box and whisker plot for the post-drive decay oscillation phase parameter (decay phase, d1) for the fit y=a1+a2*x+b1*sin(c1*x+d1)*e^{e1*x} for 1 aM RAH, 1 aM anti-RAH, and 1 aM RAH-anti-RAH from a 1 kHz post-drive decay.

FIG. 16F is a box and whisker plot for the decay coefficient for the damped oscillation envelope parameter (e1) for the fit y=a1+a2*x+b1*sin(c1*x+d1)*e^{e1*x} for 1 aM RAH, 1 aM anti-RAH, and 1 aM RAH-anti-RAH from a 1 kHz post-drive decay.

FIG. 17 is a 3D multimodal display of the optical step change (%) of 1 aM RAH, 1 aM anti-RAH, and 1 aM RAH-anti-RAH as related to the decay coefficient parameter (e1, at 100 kHz) and the magnitude of oscillation for regression (b/, at 1 kHz) as assessed from the post-drive decay fits.

FIG. 18A is a schematic of an integrated isotachophoresis (ITP) platform system according to one embodiment described herein.

FIG. 18B is a photograph of a loaded microchip fixed onto the stage of a confocal microscope, corresponding to FIG. 18A.

FIG. 19A is a schematic illustrating a wafer layer during a step of the fabrication of an ITP-functional component described herein.

FIG. 19B is a schematic illustrating a photoresist layer during a step of the fabrication of an ITP-functional component described herein, following the step of FIG. 19A.

FIG. 19C is a schematic illustrating a photomask layer during a step of the fabrication of an ITP-functional component described herein, following the step of FIG. 19B.

FIG. 19D is a schematic illustrating how the photoresist layer appears after the photomask is removed during the fabrication of an ITP-functional component described herein, following the step of FIG. 19C.

FIG. 19E is a schematic illustrating the replica molding process of polydimethylsiloxane (PDMS) around the photoresist mold during the fabrication of an ITP-functional component described herein, following the step of FIG. 19D.

FIG. 19F is a schematic illustrating the PDMS mold with a channel structure of an ITP-functional component described herein, formed by the steps of FIGS. 19A-E.

FIG. 19G is a schematic illustrating the final microfluidic channel structure of the ITP-functional component formed by the process of FIGS. 19A-E with dimensions.

FIG. 19H is a schematic illustrating the PDMS channel pattern of the ITP-functional component resulting from the process of FIGS. 19A-E.

FIG. 20A schematically illustrates a sectional view of the structure of a sensor according to one embodiment described herein. The nanopore is at the middle of the plasmonic gap.

FIG. 20B schematically illustrates a front side of the sensor (or chip) of FIG. 20A.

FIG. 20C schematically illustrates a back side of the sensor (or chip) of FIG. 20A, opposite the front side illustrated in FIG. 20B.

FIG. 21 is a schematic cross-section (profile view) of a sensor described herein.

FIG. 22 is a schematic cross-section (profile view) of a sensor described herein.

FIG. 23 schematically perspective view of a sensor described herein.

### DETAILED DESCRIPTION

Embodiments described herein can be understood more readily by reference to the following detailed description, examples, and figures. Devices and methods described herein, however, are not limited to the specific embodiments presented in the detailed description, examples, and figures. It should be recognized that these embodiments are merely illustrative of the principles of the present invention. Numerous modifications and adaptations will be readily apparent to those of skill in the art without departing from the spirit and scope of the invention.

All publications, patents and patent applications mentioned in this specification are herein incorporated in their entirety by reference into the specification, to the same extent as if each individual publication, patent, or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9.

All ranges disclosed herein are also to be considered to include the end points of the range, unless expressly stated otherwise. For example, a range of “between 5 and 10,” “from 5 to 10,” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

It is also to be understood that the article “a” or “an” refers to “at least one,” unless the context of a particular use requires otherwise.

Additionally, in any disclosed embodiment, the terms “substantially,” “approximately,” and “about” may be used interchangeably. These terms generally refer to an approximation corresponding to less than or equal to 10% variation of the stated quantity, such as plus or minus 10%, plus or minus 5%, or plus or minus 3%.

In one aspect, methods of sensing are described herein. In some embodiments, such a method comprises providing a sensor. The sensor comprises a first layer having at least one single nanohole structure or at least one dual nanohole structure, and a second layer having at least one nanopore. The single nanohole structure comprises only one nanohole. The dual nanohole structure comprises a first nanohole and a second nanohole connected by a gap. Additionally, the one nanohole (in the case of the single nanohole structure) or the gap (in the case of a dual nanohole structure) of the first layer is aligned with the nanopore of the second layer in a direction corresponding to a translocation direction across the first and second layers.

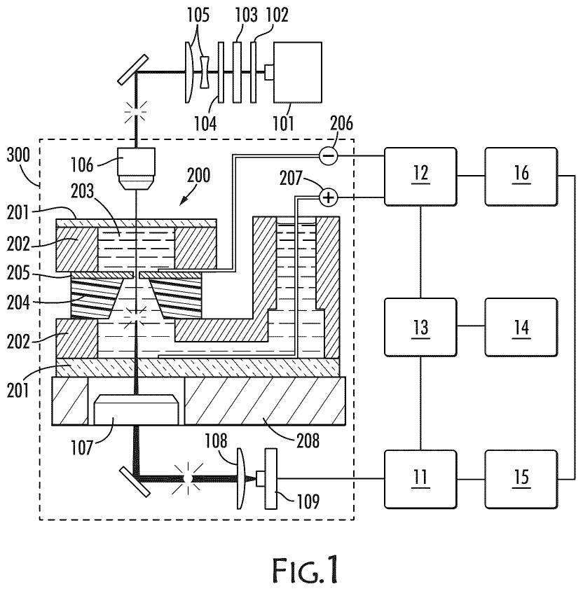

Sensors described herein having a dual nanohole structure can have any construction, structure, or property of a dual nanohole sensor described hereinbelow or described in United States Patent Application Publication No. 2020/0393456A1 by Alexandrakis et al. and directed to “Nanosensors and Methods of Making and Using Nanosensors,” the entirety of which patent application publication is hereby incorporated by reference (hereinafter referred to as “US 2020/0393456A1”). For example, in some cases, the sensor has the structure of FIG. 1 of US 2020/0393456A1. It is further to be understood that a sensor described herein can be formed using the methods described in US 2020/0393456A1 or other methods known to one of ordinary skill in the art.

Similarly, a sensor described herein having a single nanohole structure (rather than a dual nanohole structure) can likewise have any construction, structure, or property of a sensor described in US 2020/0393456A1, with the exception that the first layer comprises a single nanohole structure in place of the dual nanohole structure. Additionally, the nanohole of the single nanohole structure of such a sensor described herein can have the size and/or shape of any nanohole (e.g., a first nanohole or a second nanhole) described in US 2020/0393456A1. Other sizes and shapes are also possible. It is further to be understood that a single nanohole sensor described herein can be formed using the general methods described in US 2020/0393456A1 (modified as needed for the formation of a single nanohole structure rather than a dual nanohole structure), or using other methods known to one of ordinary skill in the art.

Turning in more detail to features of sensors described herein, a sensor comprises, in some embodiments, a chip or a wafer. The chip or wafer, in some cases, is defined by an xy-plane comprising at least a first layer and a second layer. The first layer, in some embodiments, is essentially parallel to the second layer, which is in contrast to a perpendicular z-direction. The z-direction is a translocation direction that is perpendicular and extending through the xy-plane of the chip or wafer. In some embodiments, the translocation direction goes through the first layer and the second layer of the xy-plane. The translocation direction, in some embodiments, is unidirectional, wherein the first layer is penetrated before the second layer. For example, a translocation direction can correspond to a movement through the chip or wafer from a cis chamber to a trans chamber, wherein the cis chamber is in communication with and, in some instances, partially defined by the first layer, and the trans chamber is in communication with the second layer. In some embodiments, a chip or wafer of a sensor described herein can have a substantially rectangular or square shape. In some cases, a chip or wafer can have a length and/or width of about 5-50 mm, 5-40 mm, 5-30 mm, 10-30 mm, or 10-20 mm, or about 15 mm.

In some embodiments, the first layer is positioned above or superior to the second layer. In some instances, the first and second layer are immediately adjacent layers. In some such embodiments, the first and second layers may be joined or adhered to one another via direct layer/wafer bonding. Alternatively, in other cases, the first layer and the second layer are not immediately adjacent layers but are instead spaced apart by or adhered together with one or more adhesion layers. In some such embodiments, the adhesion layer bonds to both the first layer and to the second layer with a greater bonding strength than the first layer and the second layer would bond to one another in the absence of the adhesion layer. Any adhesion layer not inconsistent with the objectives of the present disclosure may be used in a sensor described herein. For example, in some cases, an adhesion layer is formed from a metal (e.g., an elemental metal or a mixture or alloy of different metals), which may be particularly useful for adhering or bonding a metal first layer to an electrically insulating material second layer described herein, or for adhering or bonding a gold first layer to a silicon nitride second layer as described herein. In some embodiments, a metal adhesion layer can comprise titanium (e.g., elemental titanium metal) or chromium (e.g., elemental chromium metal). Other materials may also be used to form an adhesion layer of a sensor described herein. Further, an adhesion layer can have any thickness or average thickness not inconsistent with the objectives of the present disclosure. For example, in some cases, an adhesion layer has a thickness of up to 50 nm, up to 20 nm, up to 10 nm, or up to 5 nm. In some instances, an adhesion layer described herein has a thickness of about 0.5-20 nm, 0.5-15 nm, 0.5-10 nm, 1-20 nm, 1-15 nm, 1-10 nm, or 1-5 nm. In some embodiments, an adhesion layer can have a thickness of about 0.5-5 nm, 0.5-4 nm, 0.5-3 nm, 0.5-2 nm, or 0.1-1 nm.

Additionally, in some embodiments, the first and/or second layer of a sensor described herein is formed from an inorganic material, such as a metal (which may be an elemental metal or mixture or alloy of metals) or an electrically insulating material, as described further herein below.

Moreover, the first layer, in some cases, functions as an optically sensing layer. In some embodiments, as described above, the first layer is formed from a metal. Any metal not inconsistent with the objectives of the present disclosure may be used. For example, the metal can be an elemental metal or a mixture or alloy of metals. In one embodiment, the first layer is formed from gold. In some instances, a first layer described herein is formed from a different metal. The first layer material is not necessarily particularly limited. In some cases, a specific material is chosen because of its electrical conductivity properties, its chemical inertness in biological systems, and/or its compatibility with device fabrication methods described herein. In another aspect, the first layer has an average thickness of up to 500 nm in the translocation direction. In some embodiments, for example, the first layer has an average thickness of about 5-110 nm, 10-120 nm, 20-130 nm, 30-140 nm, 60-200 nm, 70-300 nm, 80-400 nm, 90-500 nm, or about 50-150 nm in the translocation direction.

Additionally, the first layer, in some embodiments, comprises at least one dual or double nanohole structure, wherein the dual or double nanohole structure comprises a first nanohole and a second nanohole. The first nanohole, in one embodiment, is essentially the same as the second nanohole. In another aspect, the nanoholes of the first layer each have an average diameter in the direction perpendicular to the translocation direction of about 80-150 nm. In other aspects, the nanoholes of the first layer each have an average diameter in the direction perpendicular to the translocation direction of about 100-150 nm, 80-100 nm, 80-120 nm, 90-120 nm, 90-130, or 100-120 nm.

In some embodiments, the nanoholes can have a center-to-center separation distance of about 150 nm or less, and in some cases, the nanoholes can overlap. For example, the nanoholes, in some embodiments, can each have a perimeter drawn by a theoretical line, thereby creating two imaginary circle-like shapes. In some instances, the theoretical lines defining the perimeter shape of each nanohole intersect in one or two locations. When the lines touch or intersect, it is understood that the nanoholes touch or overlap, respectively. In other instances, the theoretical lines defining the perimeter of each nanohole may not touch or intersect. When the lines do not touch or intersect, it is understood that the nanoholes do not touch or overlap. In some cases, the nanoholes can have a center-to-center separation distance of about 50-150 nm, 75-150 nm, 80-140 nm, 80-130 nm, or 100-120 nm.

In another embodiment, the nanoholes can have sloped or tapered interior walls along the translocation direction. The sloped interior walls, in some instances, can have a grade of about 10-30%. For example, the nanoholes can have an interior wall with a downward slope in the translocation direction such that each nanohole is shaped like an inverted cone or a funnel. In some cases, the sloped or tapered walls can have a grade of about 10-20%, 15-20%, or 15-30%.

Moreover, it is to be understood that a “nanohole” described herein can have any shape not inconsistent with the objectives of the present disclosure, including any cross-sectional shape in the xy-plane (perpendicular to the translocation direction). In some embodiments, one or both nanoholes are generally round, circular, ovoid, or ellipsoidal (ignoring any “gap” between the nanoholes, as described above). In other instances, one or both nanoholes have a triangular or other polygonal cross-sectional shape in the xy-plane. The precise shape of a nanohole described herein is not particularly limited. It is further to be understood that the size and/or center-to-center separation of a pair of nanoholes described herein can be selected based on the cross-sectional shape of the nanohole and/or based on the biomolecule analyte to be optically trapped in the dual nanohole structure. In one exemplary embodiment, for instance, two equilateral triangular nanoholes may be used having side lengths of 50-150 nm, wherein vertices of the triangular nanoholes are joined or separated by the gap of the dual nanohole structure.

For clarity, it is to be understood that a sensor described herein can have a single nanohole structure rather than, or in place of, a dual nanohole structure. In such instances, the single nanohole can have the same size, shape, and other physical characteristics as one of the nanoholes of the dual nanohole structure described hereinabove. For example, in some cases, the single nanohole of the first layer of a sensor described herein has a diameter or average size in the direction(s) perpendicular to the translocation direction of about 80-150 nm. In other cases, the single nanohole of the first layer has a diameter or an average size in the direction(s) perpendicular to the translocation direction of about 100-150 nm, 80-100 nm, 80-120 nm, 90-120 nm, 90-130, or 100-120 nm. Additionally, as an another example, the single nanohole of a first layer described herein, in some instances, can have sloped or tapered interior walls along the translocation direction. The sloped interior walls, in some instances, can have a grade of about 10-30%. For example, the single nanohole can have an interior wall with a downward slope in the translocation direction such that the single nanohole is shaped like an inverted cone or a funnel. In some cases, the sloped or tapered walls can have a grade of about 10-20%, 15-20%, or 15-30%.

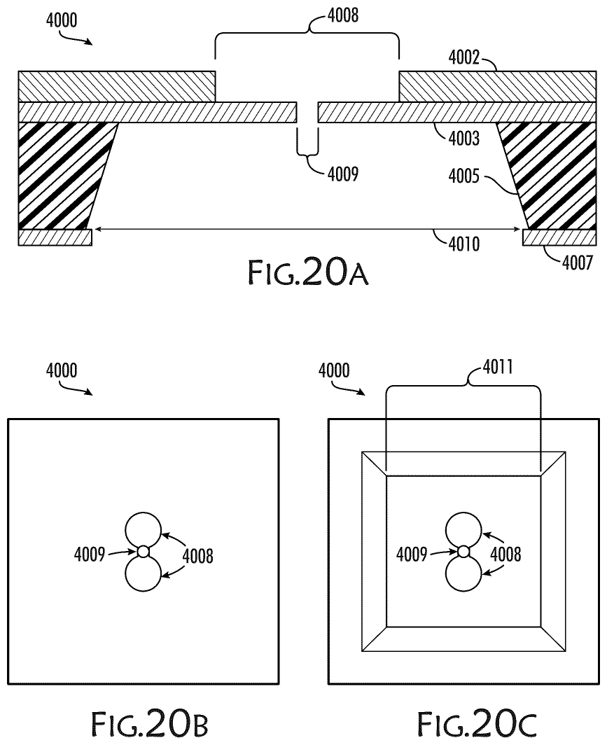

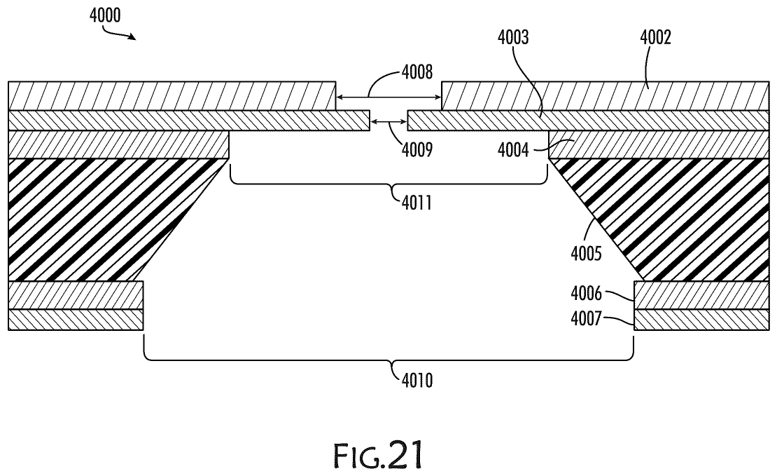

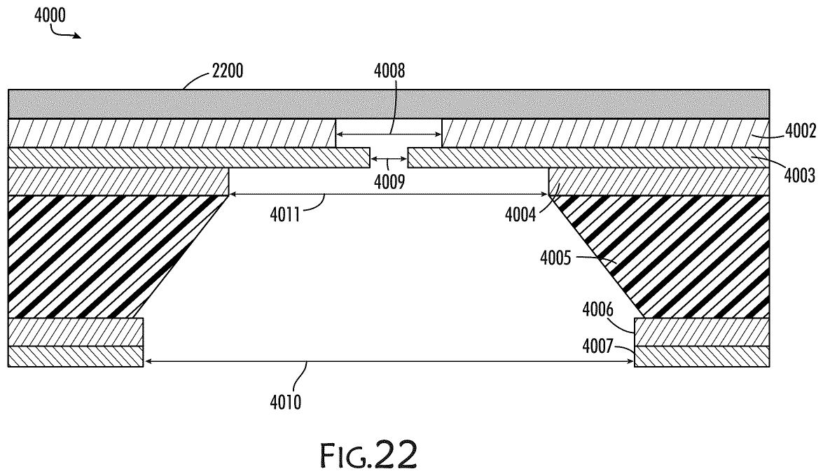

In some embodiments, the first layer can be non-continuous. As shown in FIGS. 20A-C or FIG. 21, a non-continuous first layer can comprise one or more holes and/or areas of the first layer that are etched away, removed, or generally absent, i.e., materially vacant. Such a hole, removal, or material vacancy of the first layer, in some instances, can create one or more areas of the first layer that appear as an island separated from other areas of the first layer. Alternatively, or in addition, such a hole, removal, or absence of the first layer, in some cases, can create a first layer having a Swiss cheese-like pattern (e.g., as described in US 2020/0393456A1). In some cases, where a hole or area of material vacancy in the first layer exists, the second layer can be exposed and/or visible. It should be understood that a non-continuous first layer comprising one or more holes or areas that are etched away or generally absent are in addition to the one or more dual nanoholes, and are substantially larger in diameter and area than the dual nanoholes, e.g., orders of magnitude larger. Furthermore, in some cases, an area of a non-continuous first layer having a hole and/or a material vacancy need not have necessarily had a continuous first layer followed by etching or removal. In some cases, a non-continuous first layer having holes and/or material vacancies can be formed by selective deposition of the first layer.

In some embodiments, a non-continuous first layer comprises a perimeter circumscribing the dual nanohole of the first layer, wherein the perimeter is an edge of the first layer. For example, the perimeter can define an island of the first layer that is separated from other areas of the first layer. In some embodiments, a geometric perimeter, such as circular, rectangular, or square perimeter, can be defined around the dual nanohole of the first layer. In some cases, one or more areas of the first layer outside the perimeter can be etched away or removed, such that the second layer can be visible through the first layer in the areas where it is etched away or removed, as described, e.g., in US 2020/0393456A1. In some cases, a first layer perimeter surrounding a dual nanohole structure can have a circumference measuring 1 μm-50 mm, 1 μm-40 mm, 1 μm-30 mm, 1 μm-20 mm, 1 μm-10 mm, or 1 μm-1 mm. In some embodiments, an island 803 in the first layer can have an area of about 1 μm²-100 mm². In some cases, a hole and/or island 803, as described herein that is not the one or more dual nanoholes in the first layer can have an area of about 100 μm²-100 mm², 100 μm²-80 mm², 100 μm²-70 mm². 100 μm²-60 mm², 100 μm²-50 mm², or 100 μm²-40 mm², 100 μm²-30 mm², 100 μm²-20 mm², or 100 μm²-10 mm², 100 μm²-10 mm², 100 μm²-5 mm², 100 μm²-1 mm², or 100 μm²-0.5 mm².

Not intending to be bound by theory, it is believed a non-continuous first layer having islands and/or holes, as described above, can reduce metal layer shielding of the externally applied electric field across the sensor and can increase the electrical conductivity of ionic solution added to the sensor. Consequently, it is believed that a non-continuous first layer can increase the throughput of analytes present in the ionic solution. Additionally, in some cases, such holes and/or selectively deposited areas of the first layer can act as alignment markers.

The second layer, in some embodiments, enables electrical sensing and thus functions as an electrical sensing layer. The second layer is formed from an electrically insulating material in some cases. For example, in one embodiment, the second layer is formed from a silicon nitride. Any silicon nitride not inconsistent with objectives of the present disclosure can be used. In some cases, silicon nitride comprises SiₓN_{y}. In some cases, silicon nitride comprises Si₃N₄. In some instances, a second layer described herein is formed from a ceramic material. As described above, such a ceramic material can be electrically insulating. In some embodiments, a second layer described herein is formed from a metal oxide such as a transition metal oxide. In some cases, a second layer described herein is formed from a silicon oxide such as SiO₂. Other electrically insulating materials may also be used. The electrically insulating material is not necessarily particularly limited. In some cases, a specific material is chosen because of its electrical conductivity properties, its chemical inertness in biological systems, and/or its compatibility with device fabrication methods described herein. Additionally, the second layer, in some embodiments, has an average thickness of up to 100 nm, or up to 70 nm in the translocation direction. For example, the second layer can have an average thickness of about 5-100 nm, 5-70 nm, 10-70 nm, 20-80 nm, 20-70 nm, 30-120 nm, 30-90 nm, 30-70 nm, 40-100 nm, 40-70 nm, or 50-100 nm in the translocation direction.

In addition, the second layer, in some embodiments, comprises at least one nanopore. The nanopore, in one case, has a diameter of at least 5 nm. In other cases, the nanopore has a diameter of about 2-20 nm, 5-25 nm, 15-35 nm, 20-40 nm, or 10-30 nm. In some embodiments, the nanopore is a solid-state nanopore. As understood by one of ordinary skill in the art, a solid-state nanopore is not a biological nanopore, as a solid-state nanopore comprises structural and functional differences that are distinguishable from a biological nanopore.

In some embodiments, the first nanohole and the second nanohole are connected by a gap. The gap as described herein is defined by a continuous hole or opening in the first layer connecting the first nanohole and the second nanohole. The gap, in some instances, is measurable in the x- and y-directions of the xy-plane of the chip. In some instances, the gap defines a line.

In some embodiments, the length and the width of the gap are measured in the xy-plane. In one embodiment, the width and/or length of the gap is defined by a distance between the points of intersection of the theoretical lines defining the perimeter of each nanohole. In some cases, the gap has a width and/or length of about 10-50 nm. In some embodiments, the gap has a width and/or length of about 20-50 nm, 20-40 nm, 30-50 nm, or 20-30 nm.

In some cases, the width and/or length of the gap is defined by the diameter of the nanopore. For example, in some embodiments, the width and/or length of the gap is within 10% of the diameter of the nanopore. Additionally, the gap, in some embodiments, is continuous with the nanopore in the translocation direction. The gap, in other embodiments, has a measurable width and/or length greater than the diameter of nanopore. In other embodiments, the width and/or length of the gap is less than 200% the diameter of the nanopore. In some embodiments the width and/or length of the gap is between 100% and 200% the diameter of the nanopore.

In some embodiments, the center of the gap, determined by its center point in the xy-plane, and the center of the nanopore, also determined by its center point the xy-plane, are aligned in the translocation direction. For example, in some embodiments, the center of the gap of the first layer is aligned with the center of the nanopore of the second layer in a direction corresponding to a translocation direction across the first and second layers, and the centers are spatially separated in the x- or y-direction of the xy-plane by less than 10 nm, or less than 5 nm.

For clarity, it is to be understood that a sensor described herein can have a single nanohole structure rather than, or in place of, a dual nanohole structure. In such instances, the center of the single nanohole, determined by its center point in the xy-plane, and the center of the nanopore, also determined by its center point the xy-plane, are aligned in the translocation direction. For example, in some embodiments, the center of the single nanohole of the first layer is aligned with the center of the nanopore of the second layer in a direction corresponding to a translocation direction across the first and second layers, and the centers are spatially separated in the x- or y-direction of the xy-plane by less than 10 nm, or less than 5 nm.

The sensor chip or wafer described herein, in some embodiments, further comprises an optional third layer. The presence of such a layer is preferred in some embodiments. The third layer, in some embodiments is positioned inferior or adjacent to the second layer, such that the second layer is positioned between the first and third layers. The third layer, in some embodiments, can act as an electrically insulating layer that is secondary or supplemental to the second layer, which is also an insulating layer. Thus, the third layer, in some embodiments, can be formed from an electrically insulating material. For example, in some cases, the third layer can comprise or be formed from silicon dioxide (SiO₂). In some instances, a third layer described herein is formed from an electrically insulating material described hereinabove, such as a ceramic material or transition metal oxide. Other electrically insulating materials may also be used. The electrically insulating material of the third layer is not particularly limited. In some cases, a specific material is chosen because of its electrical conductivity properties, its chemical inertness in biological systems, and/or its compatibility with device fabrication methods described herein. Not intending to be bound by theory, it is believed that, in some instances, the third layer, which can act as an insulating layer, can contain leakage or prevent the passage of current through any additional layers present beyond the second layer of the chip (in the “downward” direction in FIG. 20A or FIG. 21, for instance). In some embodiments, a third layer can have an average thickness of at least 50 nm, at least 100 nm, or at least 500 nm. In some instances, a third layer can have an average thickness of 100-5000 nm, 100-1000 nm, or 100-500 nm.

In some embodiments, the sensor chip or wafer described herein can comprise a fourth layer positioned inferior or adjacent to the third layer, such that the third layer is positioned between the second and fourth layers. Alternatively, the fourth layer can be positioned adjacent the second layer in the absence of a third layer. The fourth layer, in some embodiments, comprises or is formed from silicon. For example, in some cases, the fourth layer can be formed from pure silicon. Other semiconducting materials may also be used. In some embodiments, the fourth layer can act as a semiconducting layer. The fourth layer, in some instances, can have an average thickness of about 1-1000 μm, 10-1000 μm, 50-1000 μm, or 50-500.

In some embodiments, the sensor chip or wafer described herein can comprise one or more layers in addition to the third and fourth layers, such that the fourth layer is positioned between the second or third layer and the one or more additional layers. For example, in some instances, one or more additional layers comprising silicon, including pure silicon, silicon dioxide, and/or silicon nitride can be used. Such additional layers can have an average thickness of about 1-1000 μm, 100-1000 μm, 200-800 μm, 300-600 μm, or about 500 μm.

In some cases, the third layer, fourth layer, and/or additional layers can define a window or an opening “beneath” the first and second layers (e.g., “downward” in FIG. 20A or FIG. 21), including a window that extends through the third, fourth, or additional layers, when present. The window, in some cases provides a passage way in the translocation direction from the nanohole of the second layer into a trans chamber of the sensor. Additionally, in some instances, the one or more layers can have sloped or tapered walls in the translocation direction. The sloped walls can have a cone-like shape that taper in the opposite direction of the sloped walls of the nanoholes of the first layer. For example, a distance measured across the window opening in an xy-plane of a layer near the nanopore is smaller than a distance measured across the window opening in an xy-plane of the layer farthest from the nanopore.

One non-limiting example embodiment of a sensor or chip described herein is illustrated schematically in FIG. 20. Specifically, FIG. 20A schematically illustrates a sectional (or profile) view of the structure of a sensor according to one embodiment described herein. FIG. 20B schematically illustrates a front side of the sensor (or chip) of FIG. 20A. FIG. 20C schematically illustrates a back side of the sensor (or chip) of FIG. 20A, opposite the front side illustrated in FIG. 20B. With reference to FIGS. 20A-C, the sensor (4000) comprises a first layer (4002) comprising a dual nanohole structure (4008). The sensor (4000) also comprises a second layer (4003) comprising a nanopore (4009). The nanohole structure (4008) is aligned with the nanopore (4009) as described herein, in a direction corresponding to a translocation direction across the first and second layers (from the top of the page to the bottom of the page as illustrated in FIG. 20A). It is further to be understood that the dual nanohole structure (4008) could be replaced with a single nanohole structure as described herein (e.g., illustrated by a single circle centered over the nanopore (4009)). In the embodiment of FIG. 20A, the sensor (4000) comprises additional layers (4005, 4007), which could be third, fourth, or nth additional layers described herein. For example, in some cases, layer (4005) is formed from silicon. Moreover, in the embodiment of FIG. 20, a window (4010) is defined by the additional layers (4005, 4007).

Another non-limiting example embodiment of a sensor or chip described herein is illustrated schematically in FIG. 21, which schematically illustrates a sectional or profile view of another sensor (4000). With reference to FIG. 21, the sensor (4000) comprises a first layer (4002), such as a layer formed from gold (Au). The first layer (4002) comprises a dual nanohole structure (4008), which could have a width of 100 nm for example. The sensor (4000) also comprises a second layer (4003), such as a layer formed from a silicon nitride. The second layer (4003) comprises a nanopore (4009), which could have a width of 25 nm for example. The nanohole structure (4008) is aligned with the nanopore (4009) in a direction corresponding to a translocation direction across the first and second layers (from the left of the page to the right of the page as illustrated in FIG. 21). It is further to be understood that the dual nanohole structure (4008) could be replaced with a single nanohole structure as described herein (e.g., illustrated by a single circle centered over the nanopore (4009)). Further, in the embodiment of FIG. 21, the sensor (4000) comprises additional layers (4004, 4005, 4006, 4007). A third layer (4004) is formed from silica (for example) and defines a gap (4011) that is, for example, 100 μm wide in this embodiment, though other widths are possible. A fourth layer (4005) is formed from silicon (for example). A fifth layer (4006) is formed from silicon dioxide (for example), and a sixth layer (4007) is formed from silicon nitride (for example). The fifth and sixth layers (4006, 4007) define a window (4010). In the embodiment of FIG. 21, the thicknesses of the layers (in the translocation direction, or perpendicular to the ‘stacking’ direction of the layers) are approximately as follows: 100 nm, 50 nm, 500 nm, 525 μm, 500 nm, and 500 nm, respectively, for the first, second, third, fourth, fifth, and sixth layers. Other thicknesses and compositions of the sensor layers are also possible, as described herein.

Moreover, it is to be understood that the example embodiments of FIGS. 20 and 21 are non-limiting example embodiments, and other structures are also possible, as further described herein.

It is also possible, in some embodiments, for the sensor to include or be coupled to a layer, device, or structure for concentrating a test sample, prior to analyzing the test sample as described herein. For example, in some cases, an isotachophoretic layer, device, or structure is disposed on top of the sensor. Such a layer, device, or structure can be used to concentrate an analyte within a test sample using isotachophoresis (ITP) prior to contacting the test sample with other layers or components of the sensor (e.g., the first layer of the sensor as described above).

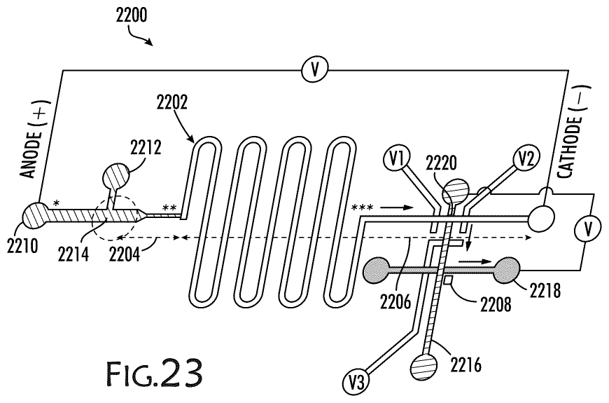

FIGS. 22 and 23 illustrate one embodiment of a sensor comprising such an isotachophoretic layer, device, or structure. FIG. 22 schematically illustrates a sectional (or profile) view of a sensor (4000). With reference to FIG. 22, the sensor (4000) of FIG. 22 has the same structure as sensor (4000) as described in FIG. 21, except an additional layer or component (2200) is disposed on “top” of the “stack” in FIG. 22, or otherwise in fluid communication with the rest of sensor (4000). Layer (2200) schematically represents, in sectional view, an ITP layer, component, or device (hereinafter referred to as the “ITP device”). The ITP device (2200) can be an integrated part of the sensor (4000), or the ITP device (2200) can be in fluid communication with the sensor (4000) without being an integral part of the sensor (4000). The ITP device (2200) may also, in some embodiments, be much larger than the sensor (4000). The ITP device (2200) is further illustrated in FIG. 23, according to one possible embodiment.

FIG. 23 schematically illustrates a perspective view of an ITP device (2200), according to one embodiment described herein. The ITP device (2200) of FIG. 23 may also be referred to as a cascade-chip or integrated ITP microfluidic device. With reference to FIG. 23, the ITP device (2200) comprises various structural features. In the embodiment of FIG. 23, the ITP device (2200) comprises an anode and a cathode having an applied voltage (V) in electrochemical communication with a isotachophoresis (ITP) separation channel (2202). The ITP separation channel (2202) is a microfluidic channel and comprises a plurality of turns or switchbacks, as illustrated in FIG. 23. Additionally, the ITP device (2200) comprises a first zone (2204) and a second zone (2206). First and second zones (2204, 2206) are spatial zones extending spatially as indicated by the double headed arrows in FIG. 23. First and second zones (2204, 2206) can also be referred to as area reduction zones, as explained further hereinbelow. In the embodiment of FIG. 23, the ITP device (2200) also comprises an intersection point (2208), corresponding to the location of a sensor described herein, such as a sensor (4000) illustrated in FIG. 20, FIG. 21, or FIG. 22.

The ITP device (2200) further comprises a first input port (2210), a second input port (2212), a T-junction (2214), a vertical feed channel (2216), and an eluate collection channel (2218). As described further herein, the vertical feed channel (2216) and the eluate collection channel (2218) intersect at the intersection point (2208).

As illustrated in FIG. 23, the portion of the ITP device (2200) marked with hatching in FIG. 23 defines a test sample input region of the ITP device (2200). This region includes or defines a microfluidic structure that differs from the sinuous ITP separation channel (2200) but leads into the ITP separation channel (2200). Together, the ITP separation channel and the microfluidic structure of the sample input region can be referred to as the overall “flow microchannel” of the device (2200).

As illustrated in FIG. 23, the cross sectional area of the flow microchannel in zone 1 is much larger than the cross sectional area of the flow microchannel in zone 2. More specifically, there is a gradual reduction of the cross section (with a width and a depth change along the channel), as seen in the dashed circle of FIG. 23. The depth change of the channel begins right after the T-junction (2214) shown inside the dashed circle. The T-junction (2214) is used to control the sample loading in the device from the first input port (2210) and the second input port (2212). The non-limiting example device shown in FIG. 23 includes a 1000 times reduction in cross-sectional area from the larger cross-sectional area region (10 mm wide x 1 mm deep) to the smaller cross-sectional area region (0.1 mm width x 0.1 mm deep).

Not intending to be bound by theory, it is believed that a large reduction (e.g., 100 to 10000 times reduction, such as the 1000 times reduction of the embodiment of FIG. 23) in cross-sectional area from zone 1 to zone 2 helps achieve a large pre-concentration factor (e.g., 100 to 10000 times, such as the 1000 times pre-concentration factor of the embodiment of FIG. 23), where the pre-concentration factor refers to the concentration of the analyte within a test sample flowed through the ITP device (2200).

Since analyte migration is proportional to current density, the voltage is lowered once the analyte/test sample enters zones 1 and 2 for proper resolution. To further improve separation efficiency, one or more electro-osmotic flow suppressors (e.g. poly(vinylpyrrolidone) or poly(ethylene glycol) species of different molecular weights) can be added to both the leading and terminal electrolyte of the ITP process. Additionally, if desired, non-detectable spacer ions can be used, as understood by one of ordinary skill in the art.

As illustrated in FIG. 23, the ITP device (2200) can be integrated with a sensor (e.g., a SANE sensor) described herein. The test sample can be introduced through the first input port (2210). More specifically, input needles (not shown) carrying the test sample and ITP separation liquid solution (also called ITP electrolyte or buffer) can be connected to the first input port (2210) and second input port (2212), respectively. One of the two input channels connected to the input ports (2210, 2212) prior to the T-junction (2214) injects ITP electrolyte or buffer with controlled timing so that the electrolytes/buffers are disposed on either side of (or ‘sandwich’) the injected volume or fraction of the test sample. In this manner, the test sample fraction or ‘plug’ is disposed between fractions, ‘plugs’, or portions of ITP buffer/electrolyte.

The test sample and ITP electrolytes/buffers are driven across or through the flow microchannel using electrical potential with valves 1 and 2 open, and valve 3 closed from a power supply (e.g., XHR 600-1, Xantrex Technology Inc., Vancouver, Canada, not shown). The two buffers/electrolytes (one preceding and one lagging relative to the analyte plug) move with different speeds under the external voltage difference applied across the entire device. As a result, the preceding electrolyte, lagging electrolyte, and analyte-containing test sample mix, and as they travel through the sinuous ITP separation channel (2202), chemical species within the mixed fluid separate according to ITP principles (e.g., based on mass due to electrophoretic mobility differences). At steady state, molecular species, such as proteins, within the test sample will separate out into discrete zones because of the difference in their electrophoretic mobilities. The targeted analyte band should occur or be found at an intersection point (2220) of the ITP separation channel (2202) and the vertical feed channel (2216) to the nanopore sensor. After analyte fractionation occurs in the ITP separation channel (2202), valves 1 and 2 are closed, valve 3 is opened, and the analyte-containing fraction from the ITP separation channel is fluidically injected into the vertical feed channel (2216), which runs to the SANE sensor (e.g., the sensor (4000) of FIG. 21 or FIG. 22). The SANE sensor is located just under the intersection point (2208) of the vertical feed channel (2216) and the horizontal eluate collection channel (2218). The ITP separation process as described herein can occur in less than a minute, less than 30 seconds, or less than 10 seconds. When the analyte-containing fraction reaches the SANE sensor, dual mode analysis (optical and electrical DC and AC) of the analyte-containing sample can occur as described further herein.

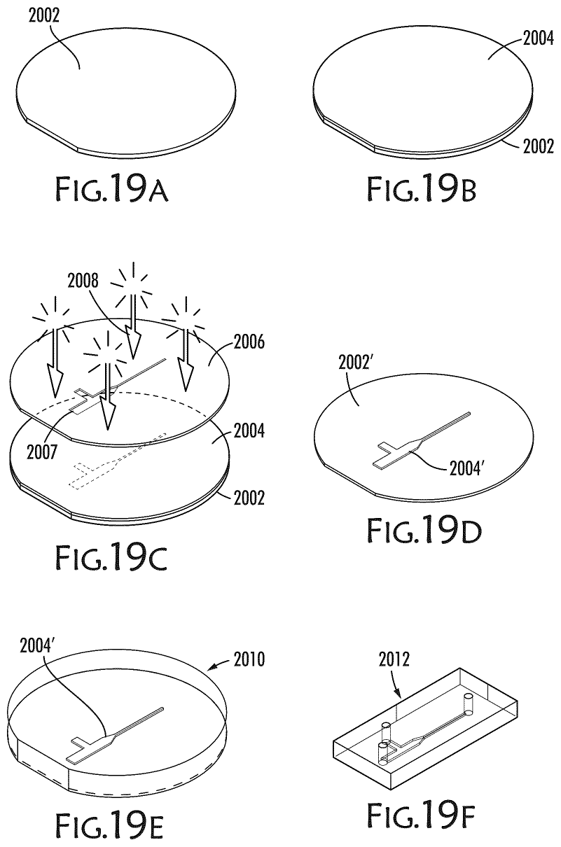

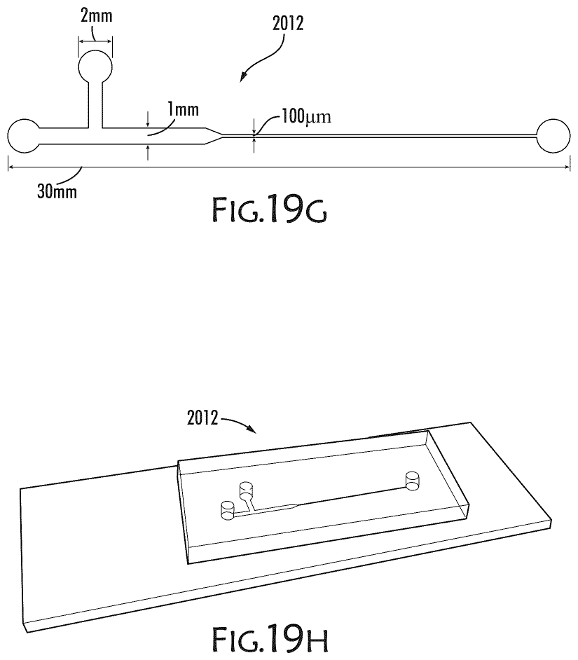

FIGS. 19A-F illustrate steps of manufacturing portions of an ITP-functional layer, device, or structure, such as the ITP device (2200) of FIG. 23. Specifically, as illustrated in FIG. 19, a test sample input region or portion of the device (2200) is fabricated. However, it is to be understood that other portions of an ITP device (2200) can be manufactured in a similar manner or using other fabrication methods understood by those of ordinary skill in the art, including using known microfluidic fabrication techniques (wherein microfluidics can refer to structures having dimensions of 500 μm or less, 100 μm or less, less than 100 μm, 50 μm or less, or 10 μm or less). With reference to FIG. 19, a photoresist (2004) is applied to a wafer (2002) (FIG. 19B). Next, UV light (2008) is applied through a photomask (2006) defining the desired structure (as shown in FIG. 19C), resulting in a patterned structure (2004′) being formed on the exposed wafer (2002′) (FIG. 19D). This structure is then used as a mold for forming a microfluidic structure (2012) in polydimethylsiloxane (PDMS) or another molding material, as illustrated in FIGS. 19E and 19F. The resulting structure is shown in FIGS. 19G and 19H.

In some implementations, an ITP microchannel structure or device described above is disposed over or in contact with the first layer of the sensor. Moreover, the ITP structure or device can be bonded or adhered to one or more other layers of the sensor. In some cases, for example, the ITP structure or device forms a unitary chip with the first layer and the second layer of the sensor.

Turning again to specific steps of methods of sensing described herein, a method described herein further comprises providing a test sample comprising an analyte and contacting the test sample with the first layer of the sensor. Particular test samples and analytes are described further hereinbelow, including the specific Examples. More generally, the test sample can be provided and contacted with the first layer of the sensor in any manner not inconsistent with the technical objectives of the present disclosure. In some embodiments, for example, the test sample is provided in a chamber positioned cis of a translocation direction of the sensor. For example, a cis chamber can be positioned adjacent and/or superior to a first layer of a chip of the sensor, as described above, such that placing or positioning the test sample in the cis chamber comprises contacting the test sample with the first layer of the sensor.

It is also possible, in some embodiments, to concentrate the test sample prior to contacting the test sample with the first layer of the sensor. In particular, in some cases, the concentration of the analyte within the test sample is increased. Such concentration of the test sample or of the analyte within the sample can be carried out in any manner not inconsistent with the objectives of the present disclosure. In some preferred embodiments, the test sample is concentrated using isotachophoresis (ITP). For example, in some implementations, the test sample is concentrated using an ITP microchannel structure, including an ITP microchannel structure described further hereinabove and hereinbelow.

Additionally, in some embodiments, the first layer of the sensor is an optically sensing layer. When the first layer of the sensor is the optically sensing layer, it is expected that the test sample is subjected or exposed to the optically sensing layer of the sensor before being subjected or exposed to other layers of the sensor.

Methods described herein also comprise irradiating the single nanohole structure or the dual nanohole structure of the sensor with a beam of electromagnetic radiation. Irradiating the nanohole structure (single or dual) can comprise irradiating with a laser beam or laser light (or other suitable electromagnetic radiation). In some cases, the laser beam can be polarized circularly and/or linearly prior to focusing on the nanohole structure. In some cases, linearly polarized light is preferred for impingement on the nanohole structure. Additionally, in some instances, the laser beam can be focused on the nanohole structure using one or more mirrors.

The wavelength of electromagnetic radiation used is not necessarily limited. In some embodiments, the laser beam or other electromagnetic radiation comprises visible light or has a wavelength (or average wavelength) centered in the visible region of the electromagnetic spectrum, such as between 450 nm and 750 nm, between 500 nm and 700 nm, or between 550 nm and 650 nm. In some cases, the laser beam or other electromagnetic radiation comprises infrared (IR) light or has a wavelength (or average wavelength) centered in the IR region of the electromagnetic spectrum. For example, in some instances, a laser beam described herein has a wavelength centered in the near-IR (NIR, 750 nm-1.4 μm), short-wavelength IR (SWIR, 1.4-3 μm), mid-wavelength IR (MWIR, 3-8 μm), or long-wavelength IR (LWIR, 8-15 μm). Moreover, in some embodiments, a laser beam or other electromagnetic radiation described herein has an emission profile/wavelength distribution overlapping with a wavelength at which water and/or biological tissue has an absorption minimum, such as a wavelength between about 700 nm and about 800 nm or between about 1.25 μm and about 1.35 μm.

In another aspect, the method of sensing comprises optically trapping the analyte in (1) the single nanohole structure or (2) in the dual nanohole structure and/or (3) in the gap of the first layer of the sensor. Optical trapping, in some embodiments, is a result of irradiating the nanohole structure of the first layer of the sensor with the beam of electromagnetic radiation. Thus, in methods of sensing described herein, a step of optically trapping the analyte is performed prior to and/or during a step of irradiating the nanohole structure. In some instances, the optical trapping lasts for at least 1 microsecond and less than 100 seconds. In some embodiments, optical trapping lasts for about 1 millisecond-60 sec, 1 millisecond-30 sec, 1 millisecond-10 sec, 1 millisecond-5 sec, 1 millisecond-5 sec, or 10 millisecond-5 sec.

In some embodiments, a step of optically trapping the analyte can comprise one or more optical trapping events. In some cases, an optical trapping event represents the optical trapping of a single analyte species, such as a single molecule. In other cases, an optical trapping event represents the optical trapping of more than one analyte species, such as more than one analyte molecule. For example, in some cases, an optical trapping event can include optical trapping of a first analyte molecule followed by optical trapping of a second analyte molecule, wherein the second analyte molecule is optically trapped with the first analyte molecule. Moreover, as one example for a particular type of analyte, it should be understood that optically trapping a first non-complexed biomolecule and a second non-complexed biomolecule is not the same as optically trapping a complexed biomolecule.

In some instances, optically trapping the analyte results in a measurable surface plasmon resonance. When a first analyte species (e.g., a first molecule) is optically trapped, a first surface plasmon resonance can be measured. When a second analyte species (e.g., a second molecule) is optically trapped with a first biomolecule, a second surface plasmon resonance can be measured. In some instances, a first plasmon resonance measured from the first optically trapped analyte species/molecule can be subtracted from the second plasmon resonance measurement to obtain information related to the second analyte species/molecule. In some embodiments, the surface plasmon resonance provides information about the mass of an optically trapped analyte such as an optically trapped biomolecule. Therefore, measuring the surface plasmon resonance, in some embodiments, comprises measuring the mass of the optically trapped analyte (e.g., biomolecule).

Thus, methods of sensing described herein, in some implementations, further comprise measuring a surface plasmon resonance of the single nanohole structure or the dual nanohole structure after optically trapping the analyte in the single nanohole structure or in the dual nanohole structure and/or in the gap of the first layer of the sensor. Additionally, in some such embodiments, measuring the surface plasmon resonance further comprises determining the mass of the optically trapped analyte.

Furthermore, in some embodiments, optically trapping comprises slowing or delaying the translocation of the analyte. Such a slowing or delaying can provide greater resolution to the sensing mechanisms of sensors described herein.

Moreover, in some cases, a method described herein further comprises applying a first electric field across the nanopore to draw one or more of the analytes into the nanopore, wherein the first electric field comprises a direct current (DC) electric field. The electric field can be applied across the nanopore in any manner not inconsistent with the technical objectives of the present disclosure. In some cases, the DC electric field is provided by placing patch clamp electrodes in the cis and trans chambers of the sensor. Thus, in some cases, applying an electric field across the nanopore can comprise applying an electric field from the cis chamber to the trans chamber of the sensor. In some embodiments, applying an electric field comprises applying a 10-1000 mV bias. In some cases, applying an electric field comprises applying a 10-500 mV, 50-50 0 mV, 100-500 mV, or about 250 mV bias.

Additionally, in some embodiments, the DC electric field is temporarily reversed. For example, in some cases, the DC electric field is applied from the trans chamber to the cis chamber. Temporary reversal of the electric field is sometimes performed to prevent clogging, or a build-up of biomolecules, at the nanopore and/or the nanohole structure. It is to be understood, however, that such reversal of the DC electric field is not the same as applying an alternating current (AC), pulsed, or modulated field, including as described hereinbelow. Instead, temporary reversal of the DC electric field is a separate step for reducing or preventing clogging of the nanopore.

Methods described herein also comprise applying a second electric field across the nanopore after applying the first electric field, wherein the second electric field comprises a pulsed, modulated, or alternating current (AC) electric field. This second field can be applied in any manner not inconsistent with the technical objectives of the present disclosure. In some embodiments, for example, the second electric field is provided by placing patch clamp electrodes in the cis and trans chambers of the sensor (and the same electrodes can be used for providing the second field as well as the first DC field; different electrodes may also be used). Additionally, as described above for the first (DC) electric field, the second electric field can be applied from the cis chamber to the trans chamber of the sensor. In some embodiments, applying the second electric field comprises applying a 10-1000 mV bias, a 10-500 mV bias, a 50-500 mV bias, a 100-500 mV bias, or about a 250 mV bias.

Additionally, the second field can have an AC, pulse, or modulation frequency of up to 1 GHz. For example, in some embodiments, an AC field of frequencies up to 1 GHz can be applied to Ag/AgCl electrodes by an external function generator and detected by Axopatch electronics. It is further to be understood that an electric field that is “pulsed” or “modulated” without necessarily being an “AC” field can be “pulsed” or “modulated” by virtue of the existence of one or more of the following: cycles or periods of “on” and “off” times; cycles or periods of “high” intensity and “low” intensity; or cycles or periods of “high” frequency and “low” frequency.” In such cases, “high” and “low” are relative terms, where the terms are relative to one another (i.e., relatively high compared to relatively low), and the differences between “high” and “low” frequency or intensity is at least 20% (based on the larger number as the denominator). It is further to be understood that “on” times refer to time periods in which the AC, pulsed, or modulated electric field is “on” or “applied,” and “off” times refer to time periods in which the AC, pulsed, or modulated electric field is “off” or “not applied.”

Moreover, it is to be understood that the second field described herein can be and preferably is applied ‘over’ the first field (the DC electric field). More particularly, in some preferred embodiments, the first field (the DC electric field) is applied continuously or substantially continuously throughout a sensing method described here. The second field (the AC, pulsed, or modulated field) is applied over or simultaneously with a ‘baseline’ provided by the first field (the DC field). In other words, in some preferred embodiments described herein, even when the external second field (the AC, pulsed, or modulated field) is not applied, a DC voltage is always applied throughout the entire method, and this DC voltage (typically up to +/−200 mV) provides the baseline on which the modulation (e.g., the AC modulation) ‘rides.’ Thus, in some preferred embodiments, the first field and the second field are both applied contemporaneously for a period of time or, in other words, the second field is applied after the first field is applied, but the first field continues to be applied even during application of the second field.

Further, it should also be noted that the AC, pulsed, or modulated waveforms of the second field may or may not be sinusoidal. In some instances, these waveforms are sinusoidal. Alternatively, in other cases, these waveforms can have any other bipolar form, such as provided by square waves or triangular waves.

As described herein, applying an electric field across the nanopore, in some embodiments, results in one or more translocation events of an analyte species (such as an analyte biomolecule). A translocation event, as described herein, comprises the entry and exit of an individual analyte species (e.g., a biomolecule analyte) through the nanopore. Moreover, in some instances, applying an electric field (first and/or second) generates a measurable current across the nanopore.

In addition, in some implementations, a method described herein further comprises measuring one or more analyte properties or other properties potentially associated with optical trapping or translocation of the analyte through the nanopore of the sensor. For example, in some cases, a method described herein further comprises measuring a change in current and/or phase across the nanopore during application of the second electric field while the analyte is optically trapped and/or during one or more translocation events of the analyte through the nanopore. As described further herein, such a measurement can, in some embodiments, provide information regarding the analyte that may not otherwise be known or detected. For example, in some cases, measuring change in current and/or phase further comprises determining a charge of a translocating analyte. Moreover, in some instances, measuring change in current and/or phase further comprises determining a dielectric constant of a translocating analyte.

In addition to the measurements of the foregoing paragraph, a method described herein can also (or alternatively) comprise measuring at least one kinetic parameter of the analyte within the nanopore after removing or turning off the second electric field. Further, in some embodiments, at least one kinetic parameter is measured while the analyte decelerates or comes to a stop while optically trapped.

Various kinetic parameters can be measured using a method described herein. For example, in some cases, the at least one kinetic parameter comprises one or more of the following well known parameters in the field of biochemistry: equilibrium dissociation constant (K_{d}), binding on-rate (kₒₙ), binding off-rate (k_{off}), and bound fraction (i.e., the fraction of analyte that is in a bound or complexed state rather than an unbound or uncomplexed state). In other instances, the at least one kinetic parameter comprises analyte size (volume), analyte charge (effective charge on the outer surface of the analyte), or analyte conformation. It is to be understood that these kinetic parameters can apply to analytes that are single molecules, molecular complexes such as protein complexes, or nanoparticles used for drug and gene delivery. Moreover, the foregoing kinetic parameters can be measured in any manner not inconsistent with the technical objectives of the present disclosure. For instance, in some embodiments, the foregoing kinetic parameters can be measured as follows:

Equilibrium dissocation constant (K_{d}) (units of molar (M)) is the inverse of Kₐ, the association constant, in which Kₐ=kₒₙ/k_{off}.

Binding on-rate (kₒₙ) is the on-rate constant measured in units of M⁻¹ s⁻¹.

Binding off-rate (k_{off}) is the off-rate constant measured in units of s⁻¹, indicative of the rate of analyte unbinding events per second.

Bound fraction is the fraction of bound protein events detected over all analyte events detected. It is equal to the number of events detected by the sensor as bound analyte divided by the total number of events detected by the sensor from bound and unbound analyte.

Analyte size (volume) is the volume of analyte represented by a sphere of equivalent volume and is measured in nm³.

Analyte charge (effective charge on the outer surface of the analyte) is the net charge surrounding the surface of the analyte, and it is expressed as units of single electron charge (e) or in Coulomb.

Analyte conformation is assessed by detecting shape changes of the analyte while trapped. If the analyte is not rigid, e.g. a protein, it can change shape dynamically while inside the optical trap of the sensor. The changes in shape (protein conformation) result in dynamic changes of both optical and electrical signals detected by the sensor. Different protein shapes scatter light, causing optical signal variability, and block the current conducting through the nanopore, inducing electrical signal variability, by different amounts.

As described herein, a method according to the present disclosure can use various parameters or measurements to detect and/or characterize an analyte of interest. For example, in some cases, a method described herein uses one or more of the following parameters or measurements to detect and/or characterize an analyte: optical data, current (e.g., across a nanopore), command voltage, conductance (e.g., ratio of current to command voltage), phase change, post-decay drive fits (e.g., intercept of regression, magnitude of oscillation, decay frequency, slope of linear drift component, decay phase, and/or decay coefficient), optical step change, trapping event counts, trapping current, and nanopore translocation current spikes.

In addition, methods described herein can be used for sensing a variety of analytes. For example, in some cases, the analyte comprises complexed and/or non-complexed biomolecules. Complexed and/or non-complexed biomolecules can include, but are not necessarily limited to, exosomes, endosomes, micelles, nucleotides, proteins, lipids, and/or carbohydrates. The biomolecules can be, in some instances, complexed with one or more secondary biomolecules. Exemplary secondary biomolecules may include, but are not limited to small molecules, nucleotides, oligonucleotides, aptamers, proteins, antibodies, lipids, and/or carbohydrates. The secondary biomolecules, in some embodiments, may be of similar origin as the complexed and/or non-complexed biomolecules. In another embodiment, the secondary biomolecules may be of different origin than the complexed and/or non-complexed biomolecules. For example, a secondary biomolecule may be derived from different species or other foreign organism. In other instances, the biomolecules can be complexed with non-biological molecules. Non-biological molecules may include, but are not limited to any kind of pharmaceutical, such as an antibody, a recombinant protein, a small molecule, or other synthetic product.

Moreover, in some embodiments, the test sample is a biological sample obtained from an animal or human subject, such as a human patient or animal patient in need of diagnosis (e.g., through detection or characterization of an analyte present in a sample taken from the human or animal patient). Some conditions, illnesses, or diseases may especially benefit from the sensing provided by methods described herein. For example, in some instances, cancer diagnosis and/or treatment can be improved using the sensors and methods of the present disclosure, as described further hereinbelow in the specific Examples. Thus, in some cases, an analyte described herein comprises a Peptide-presenting Major Histocompatibility Complex Class-I (pMHC) or pMHC component. In some instances, the analyte comprises a HLA-A2 pHMC or HLA-A2 pMHC component. In still other implementations, the analyte comprises a T-Cell Receptor-mimic (TCRm) antibody. Moreover, in some preferred embodiments, the analyte comprises a TCRm antibody against a HLA-A2 pHMC or against a HLA-A2 pHMC component.

In other instances, the analyte comprises a nanoparticle such as an inorganic nanoparticle. The inorganic nanoparticle can be a metal nanoparticle, such as a gold (Au), silver (Ag), platinum (Pt), or other nanoparticle. In other instances, the inorganic nanoparticle can be a ceramic or glass nanoparticle, such as a nanoparticle formed from silica (SiO₂) or titania (TiO₂).

Moreover, in some embodiments, a test sample described herein is provided in an ionic solution, such as a salt solution. Any ionic or salt solution not inconsistent with the objectives of the disclosure can be used, including NaCl, KCl, or CaCl₂) solution.

In some embodiments, methods of sensing described herein provide for detecting or sensing analytes (such as biomolecules) at a milli-(10-3), micro-(10-6), nano-(10-9), pico-(10-12), femto-(10-15), or atto-(10-18) molar concentration of the analytes (e.g., biomolecules).

Some features and characteristics of sensors and methods of sensing according to the present disclosure are described in further detail in the specific Examples below.

### Example 1

### AC-Based Discrimination of Nanoparticles using a SANE Sensor

### A. General

Nanopores can be used to discriminate among analytes through the analysis of changes in conduction current profiles during translocation. Nanopore measurements can enable the discrimination between single molecule species in solution and can help achieve low-cost and label-free DNA sequencing. Other additional possible applications are expanding rapidly. However, the translocation times of analytes through a traditional nanopore are extremely fast, which limits the fidelity of electrical data that can be collected. Through the use of optical trapping enabled by the self-induced back-action (SIBA) effect, nanopores can be enhanced not only by slowing down the translocation of analytes but also by introducing new dimensionality to the collected data through the collection of optical data simultaneously with electrical data.

Herein, the present inventors describe a SIBA actuated nanopore electrophoresis (SANE) sensor, effectively a nanopore with plasmonic optical trapping, that has been shown to be capable of trapping individual nanoparticles, proteins and protein complexes and through the use of bimodal optical and electrical data, discriminating between analyte species. The present Example combines driving the SANE sensor with an AC voltage (or other modulated or pulsed electric field), which was previously inaccessible, mostly because of fast translocation times that are typically in the hundreds of us, which would necessitate a MHz driving frequency that exceeds the available frequency limit. The optical trap of the SANE sensor provides a trapping duration in the seconds range, which allows for frequencies as low as 1 Hz. An upper bound of possible AC measurement frequencies can be set by the amplifier hardwired filters (100 kHz) and the data acquisition sample rate (500 kHz).

Not intending to be bound by theory here (or elsewhere throughout this application), it is believed that by introducing modulation, the mobility of ions in the fluid around analytes depends on the analyte surface charge and the material properties in the case of nanoparticles, thus possibly enhancing the capacity of the sensor to distinguish between analyte species. This disclosure presents results of a new sensing method able to discriminate between 20-nm SiO₂ and 20-nm Au nanoparticles using electrical measurements. By applying a DC command voltage with a superimposed AC frequency sweep, while keeping the nanopores optically trapped in the vicinity of the nanopore's entrance, SiO₂ and Au nanoparticles were found have distinctly different electrical responses. This disclosure demonstrates the feasibility of performing these AC (or other pulsed or modulated) measurements with a plasmonic nanopore.

### B. Methods

### SANE Sensor Setup

The setup used for the method, including a laser diode, optics to polarize the laser beam, the sensor setup, the AC- and DC-generating devices, and the data acquisition instruments, are herein provided in a graphical schematic (FIG. 1). A 820 nm near-infrared laser diode (101) is used with its polarization adjusted by a quarter-wave plate (QWP) (102), a Glan-Thomson Polarizer (GTP) (103), a half-wave plate (HWP) (104), and 4× beam enhancer (4×BE) (105) together to match the orientation of the sensor's narrow waist, where plasmonic enhancement is the strongest. A mirror is used to reflect the light to the sensor. The sensor (referred to as 200 in its entirety) comprises a bottom glass layer (201), a 2 mm-thick polydimethylsiloxane (PDMS) flow cell (202) containing a KCl electrolyte solution (203), a silicon layer (204), a Au layer with a nanopore (205), and another layer of PDMS (202) topped with a glass coverslip (201). The sensor also contains Ag/AgCl electrodes (206 and 207, respectively) connected to an Axopatch 200B system (012). The sensor is positioned between a Carl Zeiss 1.3 N.A. 63× objective lens (OL) (106) and a condenser lens (CL) (107) on a Piezo stage (208). Optical data are focused with a lens (108) collected by a photodiode (109) that records the transmitted light intensity, which increases in a stepwise manner when a nanoparticle is trapped or decreases in a stepwise manner when a nanoparticle is translocated through the sensor. These data are amplified by an amplifier (11). The Carl Zeiss lens, the condenser lens, the sensor positioned between these lenses on a Piezo stage, and the parts of the photodiode that collect the optical data are all contained within a Faraday cage (300, indicated by dashed lines). Electrical data showing a positive current spike when the nanoparticle enters the optical trap, current fluctuations while the particle stays in the trap, and a negative spike when the nanoparticle escapes the trap and translocates through the immediately underlying nanopore are collected synchronously using an Axopatch 200B system (Molecular Devices, San Jose, CA) (12). Optical data and electrical data are both digitized by an Axon Digidata 1440 ADC (Molecular Devices) (13) and go to a personal computer (14) for data logging.

For the AC measurements, the optical data are also sent to an Arduino Uno (15) (Adafruit Industries, New York, NY) programmed to perform edge detection, which is to be understood as that it looks for an optical step change indicative of a trapping event that is accompanied by a simultaneous current spike in the electrical data stream. The Arduino then sends a trigger to an Agilent 33250A Waveform/Function Generator (16) (Agilent Technologies, Santa Clara, CA) that generates a user-defined pulse at a selected frequency and amplitude. The function generator then sets the command voltage for the Axopatch 200B, which is operated in voltage clamp mode. The ratio of the measured current amplitude to command voltage, i.e. conductance, is then computed as a characteristic parameter of the nanoparticle response to the AC modulation. This is referred to herein as an AC burst event. Further, the fast-Fourier transform (FFT) analysis of the recorded AC signal responses enables the calculation of the phase change while the particle is driven by the AC burst. Additional data types are obtained from fitting post-drive decay data once the driving burst has stopped to an empirically-derived formula incorporating a damped oscillation term, as described below. The DC voltage is consistently on and kept at 100 mV (−ve cis to +ve cis).

A baseline AC response is established using a model cell reference block provided by the Axopatch 200B manufacturer for calibrating the system. A model cell bath that has an equivalent circuit of a 10 MΩ resistor in series with a 4 pF capacitor is used for impedance matching during these baseline measurements with the Axopatch 200B. A baseline is taken with the sensor for the AC measurements using 40 μL of 0.3 M KCl solution at 7.4 pH. Baseline measurements are performed at 110 mV command voltage with one of the following AC frequencies superimposed: 1, 2, 5, 10, 20, 50, 100, 1000, 2000, 5000, 10000, 20000, 50000 and 80000 Hz.

The amplitude of the waveform at each frequency is set to ensure there was a high signal-to-noise ratio but low enough to ensure the Axopatch 200B does not saturate while recording the current response. 1 Hz measurements are taken with a 10 Vp-p signal, and 1 kHz are generally collected at 50 mV p-p. The Axopatch 200B front-switched command voltage port is used to connect to the function generator. This port reduces all signals by a factor of 20. Each frequency is set to pulse 5 times with 10 cycles each to enable testing the reproducibility of the response. In addition, signal decays recorded at the end of each burst are analyzed to generate additional data types for the characterization of nanoparticles.

### Nanoparticle Testing

All experiments were performed with a single sensor to allow for easy comparison among different experiments. To generate the optical trap and nanopore parts of the sensor, Ne/He focused ion beam milling was performed. 20+4-nm SiO₂ nanoparticles (MEL0010, NanoComposix, zeta potential=−40 mV) and 20±2-nm Au nanoparticles (C11-20-TM-DIH-50, Nanopartz, zeta potential=−15 mV) were assessed with this method. The SiO₂ nanoparticles were used to assess the system's response to dielectric materials. The Au particles were used to show the response of the system to conductive materials, and their responses were expected to be different. The SiO₂ nanoparticles were tested at the same frequencies as the baseline measurement for comparison with the empty trap response and model cell response. Post-decay analyses were also performed after driving the nanoparticles in the trap at a single frequency of 100 Hz. The Arduino was programmed to trigger an AC burst on both a positive and negative optical step change to ensure a trapping event AC burst was paired to a non-trapping burst event.

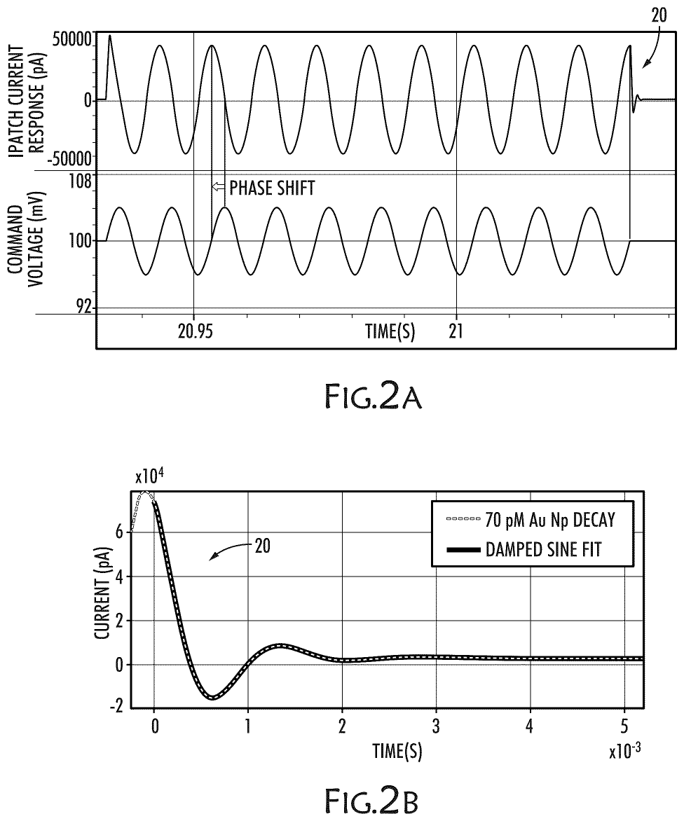

Once the data were collected, each pertinent AC burst event was noted for start and stop times and if it took place during a trapping event. The event parameters were imported into a MongoDB document database (MongoDB Inc, New York, NY) and then loaded into MATLAB (MathWorks, Natick, MA) to be processed first for a frequency response and then for a decay response. The axon binary file (.abf) generated by the pCLAMP software (Molecular Devices) was trimmed according to the event times, and a FFT was performed on the current response and command voltage of the .abf data, as depicted in FIG. 2. The center frequency of the oscillation was determined by the FFT and was then used to identify the phase shift between the command voltage and the current response. The magnitudes of the peak amplitude with both the current response and command voltage were divided to calculate the conductance of the sensor during the AC burst event. The phase change at each frequency for the model cell was subtracted from the empty trap to determine the sensor-specific phase change. The conductance at each frequency for the empty trap was divided by the model cell conductance to produce a ratio of conductance to calculate the conductance response specific to the sensor. In a similar manner, the phase change of the empty trap was subtracted from the phase change of the 1 fM SiO₂ nanoparticle solution at each frequency to look at the phase change relative to the empty sensor response. The conductance ratio was calculated at each frequency by dividing the sensor conductance for the 1 fM SiO₂ nanoparticle solution by the empty trap conductance.

The data were processed for their post-drive decay profiles. The updated event parameters were loaded back into MATLAB in which the pCLAMP data was trimmed at the termination of the AC pulse by smoothing the command voltage and then taking the first derivative to determine where along the time-axis the signal returned to the DC baseline voltage. The event time was then extended by 3 ms. Using the “prepareCurveData( ) and “fit( ) functions in MATLAB with the “NonlinearLeastSquares” option, a fit of the decay profile was created using Equation 1:

$$y = a 1 + a 2 * x + b 1 * sin ( c 1 * x + d 1 ) * {e}^{e 1 * x} . \qquad Eq . 1$$

The form of this equation was selected empirically as the sum of a damped oscillation, representing the particle's decaying oscillation, and a line with a negative slope, representing particle drift. The initial fitting parameters were set accordingly as follows: a1 and a2 were set to the minimum absolute value of the trimmed data segment, b/was set to the maximum absolute value of the trimmed data segment, c/was set empirically to 15000 for SiO₂ nanoparticles and 10000 for Au nanoparticles and the empty trap, d1 was set to 0, and e1 was set empirically to 0.653. The R2 value of the fit was used to filter out poorly fitting data, with the threshold set at 0.9, and the remaining parameters were analyzed to see how the nanoparticle type and concentrations would affect the post-drive decay parameters.

### C. Results

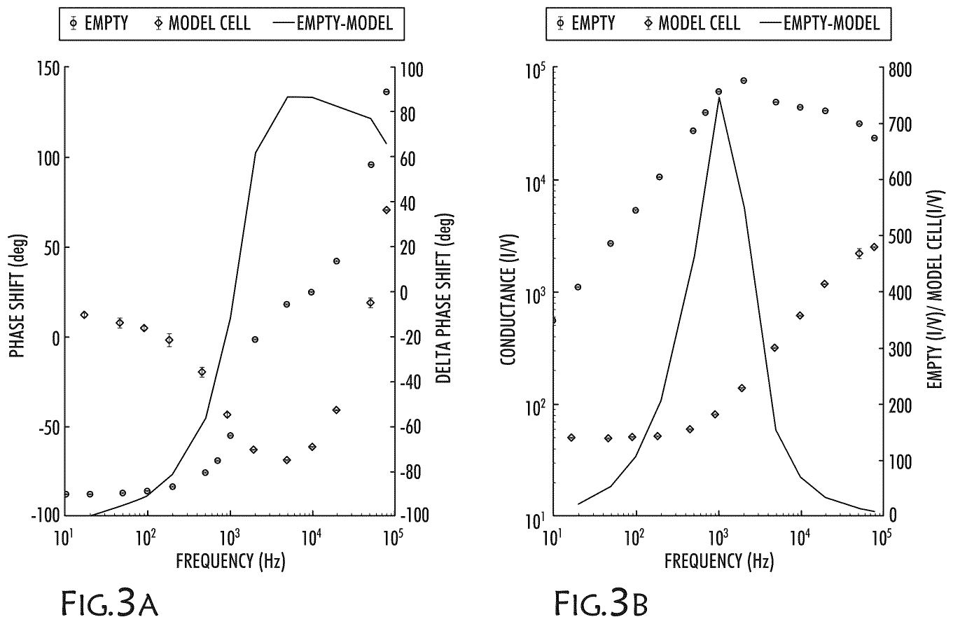

A baseline measurement of the model cell was performed. This was then repeated with the SANE sensor loaded with 0.3 M KCl and an empty trap. The results of the baseline experiments are presented in FIG. 3. Although optical transmission appeared unaffected by the AC drive (optical data not shown), significant phase and conductance changes were noted as a function of frequency. After subtracting the model cell phase response from that of the empty sensor, a biphasic phase dependence became obvious with a point of inflection around zero net phase change for the sensor, at approximately 1 kHz. Additionally, once the model cell conductance response was divided out from the empty sensor, a peak in empty sensor conductance was also noted at approximately 1 kHz. The phase shift leveled out around 5 kHz and then started decreasing with increasing driving frequency, while the conductance ratio dropped back with a point of inflection around 2 kHz. A pre-peak point of infection was also noted at approximately 200 Hz.

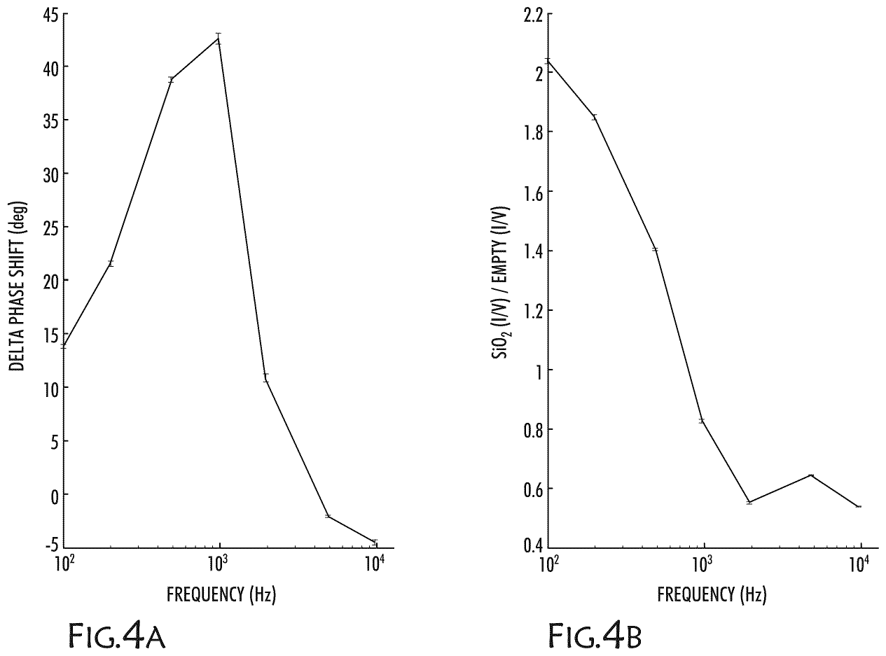

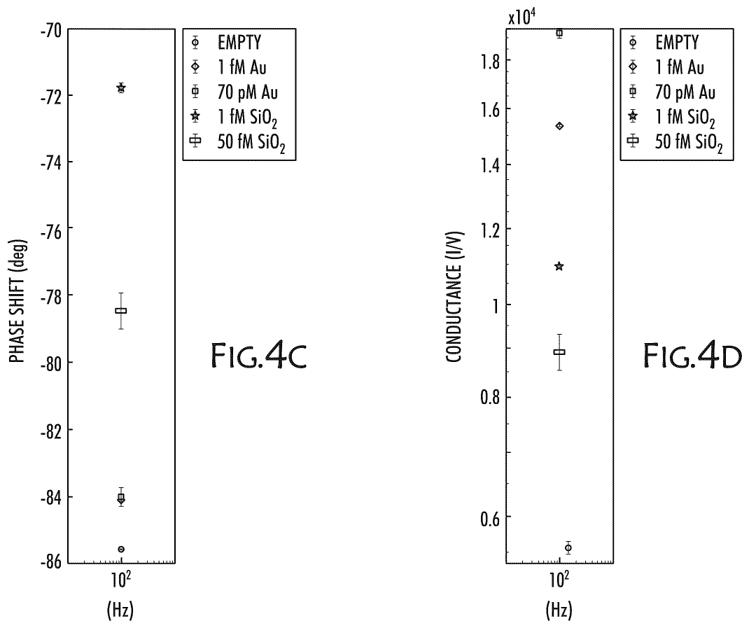

In order to avoid multi-particle interactions in the vicinity of the optical trap, ultra-low concentrations of SiO₂ nanoparticles (1 fM) were used at the same frequencies used for the baseline and compared to the response of the empty trap corrected for model cell response. The largest change in phase was seen at 1 kHz (FIG. 4A), which coincided with the point of inflection in the phase change increase of the empty trap near its zero-phase response, as shown in FIG. 3A. The relative conductance decreased with driving frequency with a point of inflection around 1 kHz, where the nanoparticle phase response was at a maximum. In addition, a secondary maximum was seen at approximately 5 kHz (FIG. 3B).

In addition to doing a frequency scan, both SiO₂ and Au nanoparticles were driven at 100 Hz to compared their relative phase shift (FIG. 4C) and conductance (FIG. 4D) responses relative to the empty sensor response at two different concentrations. The Au nanoparticles increased the conductance of the sensor chip, which also increased at the higher concentration. In contrast, the SiO₂ conductance increase relative to the empty sensor was lower and decreased with increasing SiO₂ nanoparticle concentration. In regards to the phase change relative to the empty sensor, the SiO₂ dielectric nanoparticles showed a stronger response than the conducting Au particles, which had values near to those of the empty sensor. The phase shift of the Au particles appeared to show no strong concentration dependence, whereas the SiO₂ nanoparticles showed a strong one (FIG. 4D). However, when assessed using a Wilcoxon rank sum test, a p-value of 0.002 was obtained. Therefore, at a p≤0.05, the mean phase shift for the two Au nanoparticle solutions did have a concentration-dependent phase shift.

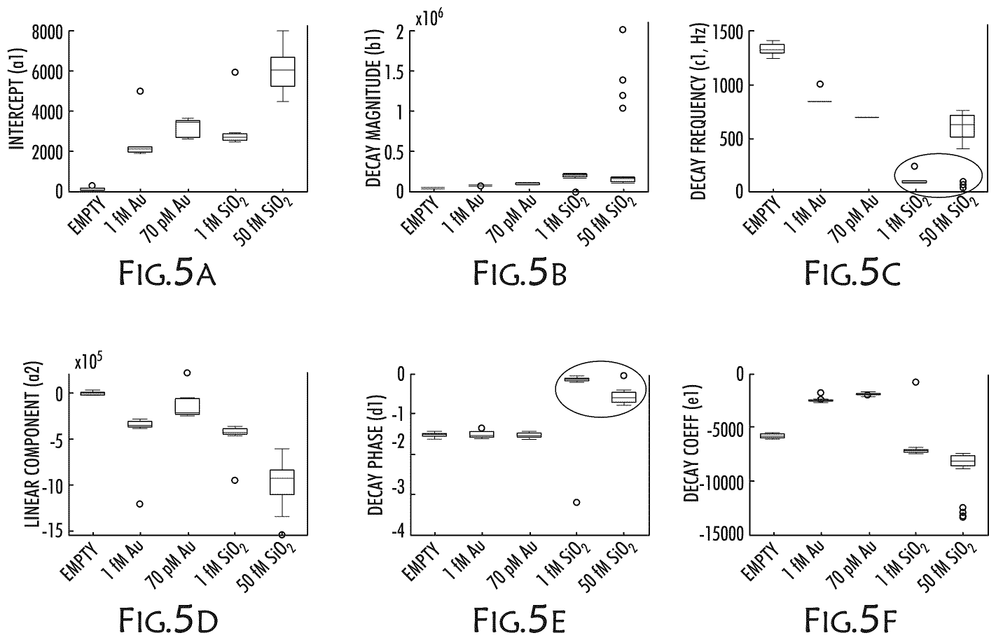

Subsequently, the post-drive decay that was fitted to damped oscillation riding on a linear slope according to Eq. I was assessed. Any fit with an R2 value less than 0.9 was excluded from the analysis to mitigate the effect of noisier measurements on fit parameters. The 100 Hz pulse post-drive decay parameters of the fit to Eq. 1 are presented in FIG. 5. FIGS. 5A and 5D show that the intercept and slope of the linear component of the drift have both particle-type and concentration-based dependence, respectively. In contrast, the empty trap had zero-intercept and a flat slope, indicating that any post-drive linear drift was the result of the nanoparticles' interactions with the applied AC bias. FIG. 5B shows the magnitude of the periodic component of the damped oscillation. This factor was dependent on the analyte type. However, the amplitude of the oscillation was largely independent of the concentration of each nanoparticle type. Further, there were several outliers in the 50 fM SiO₂ run, shown in FIG. 5B with R2 values above 0.9.

FIG. 5C shows the frequency of the post-drive damped oscillation, which was much higher than the driving frequency of 100 Hz. The empty sensor had the highest natural decay frequency, while the loaded sensor measurements showed both a nanoparticle type- and concentration-dependence at that frequency. The Au nanoparticles had a higher frequency than the SiO₂ nanoparticles and were closer to the decay frequency of the empty sensor than SiO₂ nanoparticles. Additionally, the relationship between the decay frequency and the concentration of Au nanoparticles was decreased, while the decay frequency increased with increased concentration of SiO₂ nanoparticles. The outliers of the 50 fM SiO₂ group in FIG. 5C have similar frequency with the main group of frequency responses for the 1 fM concentration (FIG. 5C, oval).

FIG. 5E shows the phase response of the damped oscillation. The response of Au nanoparticle solutions coincided with that of the empty trap, which is also formed from Au. In contrast, the SiO₂ nanoparticles had a distinct phase shift and also showed a decreasing phase shift with increasing concentration. Similar to the decay frequency, the outliers of the higher SiO₂ concentration solution correlated with the group-wise values of the lower concentration solution (FIG. 5E, oval). Finally, FIG. 5F shows the decay exponent for the envelope of the damped oscillation. The empty trap response was between the response of the two nanoparticle solutions. The concentration dependence was not as pronounced, but there were distinct differences between the particle types.

### D. Discussion

In this Example, a SANE sensor was used with an AC voltage to optically trap SiO₂ and Au particles, and after an AC burst at 100 Hz, the post-drive decay data profiles were fit using a damped oscillation model. The function selected to fit the post-drive decay (Eq. 1) was selected empirically by the inspection of curves similar to the one shown in FIG. 2A. The addition of the linear component brought SiO₂ fits from an 0.85 R2 value to above 0.9, with many reaching 0.99. The initial guesses for the fitting parameters were selected empirically to provide consistently good quality fits and are shown below in Table 1. Some sensitivity in fitting the results to initial guesses was given.

TABLE 1
Initial parameters used to fit Eq. 1.

| Parameter | Empty | Au | 50 fM SiO₂ | 1 fM SiO₂ |
|---|---|---|---|---|
| a1 | Signal min | Signal min | Signal min | Signal min |
| a2 | Signal min | Signal min | Signal min | Signal min |
| b1 | Signal max | Signal max | Signal max | Signal max |
| c1 | 10000 | 10000 | 11636 | 5000 |
| d1 | 0 | 0 | 0 | 0 |
| e1 | 0.635 | 0.635 | 0.635 | 0.635 |

The baseline analysis of FIG. 3 suggested the presence of a resonant response for the empty sensor that was reminiscent to that of a resistive-capacitive circuit, with 1 kHz as the resonant frequency. The SiO₂ nanoparticle frequency response (FIG. 4A) confirmed that a driving frequency in the vicinity of 1 kHz would be near this specific SANE sensor's highest sensitivity response. In that frequency range, the phase difference between the SiO₂ nanoparticle and the empty sensor was the largest. The sensor's phase response was near zero at that frequency, which defined the point of inflection between the negative and positive phase responses (FIG. 3A). In contrast, although the empty sensor had a conductance maximum in the 1 kHz range (FIG. 3B), the presence of SiO₂ nanoparticles acted as a low-pass filter on the sensor (FIG. 4B). The minimum seen in FIG. 4B may be a local or a global minimum.

For the driven oscillation at 100 Hz, the highest conductance was seen with the Au nanoparticles, and the conductance increased with Au nanoparticle concentration. In contrast, the SiO₂ nanoparticles showed a lower conductance than the Au nanoparticles but still higher than that of the empty sensor. The difference in conductance caused by Au and SiO₂ nanoparticles can be explained by the fact that Au is a conductor that would oppose changes in the applied voltage bias, which would, in turn, cause ionic fluid motion around these particles. Additionally, SiO₂ nanoparticles are dielectric, causing them to polarize in the presence of an AC field, which would augment ionic charge displacement relative to an empty sensor but less so than the conducting Au nanoparticle. Another factor to consider is that particles are likely to accumulate over the SANE sensor because they are driven there by the DC bias; however, they cannot easily tunnel through the optical trap. This results in a “traffic jam” of nanoparticles that may all feel the effects of the applied AC bias. Not intending to be bound by theory, it may be for this reason that the phase response for SiO₂ nanoparticles in FIG. 4D is lower at the higher concentration, as nanoparticles may experience different field strengths, depending on their position and may collide with each other, which would contribute to the decoherence of phase. On the other hand, the Au nanoparticles resist the applied AC bias near-instantaneously and collectively increase the detected conductance at higher concentrations (FIG. 4D).

Next, in respect to the SANE sensor-derived parameters from the post-drive signal decay profiles, the larger amplitude line intercept and the steeper linear slope superimposed onto the post-drive decay at 100 Hz (FIGS. 5A and 5D, respectively) could provide some insight into the charging effects of the SiO₂ nanoparticles. Once the driven oscillation has ceased at the peak point of the AC oscillation, the SiO₂ nanoparticles may hold a surface charge that needs to decay back to the DC baseline state. This effect is more pronounced at higher SiO₂ nanoparticle concentrations, as the particles that had accumulated near the mouth of the pore and all others above it contributed to different degrees, depending on their position, to this discharging effect. In contrast, Au nanoparticles did not have this sloping response because they could adjust their surface to the applied field very quickly as conductors.

The decay magnitude in FIG. 5B likely provides similar insight as the intercept (FIG. 5A) and hints at the higher charge stored on the SiO₂ nanoparticles at the peak of the AC drive. The decay frequency of post-drive oscillations shown in FIG. 5C demonstrates natural decay frequencies for the empty sensor and all nanoparticles that were much higher than the 100 Hz driving oscillation. Also, a clear dependence on nanoparticle concentration was shown. It is interesting that the decay frequency for Au nanoparticles was somewhat lower for the higher concentration, whereas in the case of SiO₂ nanoparticles, the decay frequency was higher for the higher concentration. Again not intending to be bound by theory, this phenomenon may be related to charging effects. Au nanoparticles can adjust quickly to the external field, and the interaction of the nanoparticle inside the optical trap with nanoparticles above it could force the particle to move like a “heavier particle,” which would result in a lower resonant frequency, as per Hooke's law. However, SiO₂ nanoparticles are charged up at the end of the driving cycle, which may affect the balance among the electrophoretic, dielectrophoretic, electroosmotic and optical trapping forces, which, in turn, affects the stiffness of the apparent spring constant for this trap. Another possibility related to a concentration-dependent effect is a packing effect. Ludwig et. al. observed two different performance scaling laws depending on the packing density of SiO₂. It is likely that the localized high concentration observed by Peri et. al. is occurring near the trap in the 50 fM SiO₂ solution, resulting in the dense packing of SiO₂ nanoparticles, causing the difference in the frequency response from the 1 fM SiO₂ solution.

FIG. 5E is an illustration of what was seen in FIG. 4D applied to a different harmonic motion. The SiO₂ nanoparticles have a much different phase shift than that of the Au nanoparticles, which have a concentration-independent response that is very close to the empty sensor one. The lower concentration of SiO₂ nanoparticles shows a larger phase shift than the higher concentration, lending support to the idea that the higher concentrations of SiO₂ nanoparticles reduces their oscillatory coherence by physical collisions. Of note, the outliers of the 50 fM concentration match the phase shift of the 1 fM concentration, suggesting a localized concentration effect is occurring.

Additionally, the decay coefficient in FIG. 5F serves to control as the damping envelope to the post-drive decay. The decay coefficient of Au nanoparticles is less negative than that of the empty sensor, whereas the SiO₂ nanoparticles have a more negative one. It may be that the conducting Au nanoparticles resist applied field changes and therefore are less affected by charging effects in their immediate environment as they come to a rest. On the other hand, SiO₂ particles are charged when the post-drive cycle begins, and this engenders the presence of the electroosmotic forces that would oppose translocation with a magnitude that decreased as the nanoparticles are discharged.

The optical performance of the system did not provide any insight other than there was no optical response to the AC modulation of analytes. However, this does provide some insight into the stiffness of the optical trap and the direction of motion the particles experience. The majority of optical transmission change seen by the SANE sensor is due to particles entering and leaving the trap. When a particle enters the trap, it serves as a dielectric lens and increases the intensity of light that is transmitted through the pore. If the AC modulation is causing a particle to oscillate in line with the nanopore without leaving the trap, it would likely not cause a noticeable change in the optical transmission. Neumeier et al. states that the time required for a particle to return to its favored state in the optical trap is on the order of pico-seconds. However, the particle must make its way through the trap in order for it to translocate and leave the trap. This could allow for the inline movement of the nanoparticle while in the trap. No oscillations occurred that conclusively resulted in a trapping or translocation event; therefore, the force preventing a nanoparticle from leaving the trap was likely greater than the force of the driven oscillation.

### E. Conclusions

This Example presents data for the use of a SANE sensor for AC-, pulsed-, or modulation-driven plasmonic nanopore sensing. The disclosed results in this Example show that the AC method used with the SANE sensor could discriminate between Au and SiO₂ nanoparticles of the same diameter. The model-deduced oscillation parameters during post-drive decay of the AC bursts both appeared to be concentration-dependent. At the lower concentrations used for each particle type, the difference in values between particle types for a given oscillation parameter became more pronounced. These types of AC measurements can be useful for the characterization of biological nanoparticles, such as liposomes, gene therapy vehicles, and drug delivery particles.

### F. References

1. Lu, Y., Wu, X.-Y., Ying, Y.-L., and Long, Y.-T., “Simultaneous single-molecule discrimination of cysteine and homocysteine with a protein nanopore,” Chemical Communications 55 (63), 9311-9314 (2019).
2. Jain, M., Olsen, H. E., Paten, B., and Akeson, M., “The oxford nanopore minion: delivery of nanopore sequencing to the genomics community,” Genome biology 17 (1), 1-11 (2016).
3. Lee, J. S., Oviedo, J. P., Bandara, Y. M. N. D. Y., Peng, X., Xia, L., Wang, Q., Garcia, K., Wang, J., Kim, M. J., and Kim, M. J., “Detection of nucleotides in hydrated ssdna via 2d h-bn nanopore with ionic-liquid/salt-water interface,” Electrophoresis 42 (7-8), 991-1002 (2021).
4. Nehra, A., Ahlawat, S., and Singh, K. P., “A biosensing expedition of nanopore: a review,” Sensors and Actuators B: Chemical 284, 595-622 (2019).
5. Lee, J. S., Saharia, J., Bandara, Y. N. D., Karawdeniya, B. I., Goyal, G., Darvish, A., Wang, Q., Kim, M. J., and Kim, M. J., “Stiffness measurement of nanosized liposomes using solid-state nanopore sensor with automated recapturing platform,” Electrophoresis 40 (9), 1337-1344 (2019).
6. Raza, M. U., Peri, S. S. S., Ma, L.-C., Iqbal, S. M., and Alexandrakis, G., “Self-induced back action actuated nanopore electrophoresis (sane),” Nanotechnology 29 (43), 435501 (2018).
7. Verschueren, D., Shi, X., and Dekker, C., “Nano-optical tweezing of single proteins in plasmonic nanopores,” Small Methods 3 (5), 1800465 (2019).
8. Al Balushi, A. A. and Gordon, R., “Label-free free-solution single-molecule protein-small molecule interaction observed by double-nanohole plasmonic trapping,” ACS photonics 1 (5), 389-393 (2014).
9. Peri, S. S. S., Sabnani, M. K., Raza, M. U., Ghaffari, S., Gimlin, S., Wawro, D. D., Lee, J. S., Kim, M. J., Weidanz, J., and Alexandrakis, G., “Detection of specific antibody-ligand interactions with a self-induced back-action actuated nanopore electrophoresis sensor,” Nanotechnology 31 (8), 085502 (2019).
10. Peri, S. S. S., Sabnani, M. K., Raza, M. U., Urquhart, E. L., Ghaffari, S., Lee, J. S., Kim, M. J., Weidanz, J., and Alexandrakis, G., “Quantification of low affinity binding interactions between natural killer cell inhibitory receptors and targeting ligands with a self-induced back-action actuated nanopore electrophoresis (sane) sensor,” Nanotechnology 32 (4), 045501 (2020).
11. Storm, A. J., Storm, C., Chen, J., Zandbergen, H., Joanny, J.-F., and Dekker, C., “Fast dna translocation through a solid-state nanopore,” Nano letters 5 (7), 1193-1197 (2005).
12. Saharia, J., Bandara, Y. N. D., Karawdeniya, B. I., Hammond, C., Alexandrakis, G., and Kim, M. J., “Modulation of electrophoresis, electroosmosis and diffusion for electrical transport of proteins through a solid-state nanopore,” RSC advances 11 (39), 24398-24409 (2021).
13. Zehtabi-Oskuie, A., Jiang, H., Cyr, B. R., Rennehan, D. W., Al-Balushi, A. A., and Gordon, R., “Double nanohole optical trapping: dynamics and protein-antibody co-trapping,” Lab on a Chip 13 (13), 2563-2568 (2013).
14. Morton, J. J., “Electrical and optical properties of materials,” (2009).
15. Ludwig, M., Witt, M. U., and von Klitzing, R., “Bridging the gap between two different scaling laws for structuring of liquids under geometrical confinement,” Advances in colloid and interface science 269, 270-276 (2019).
16. Neumeier, L., Quidant, R., and Chang, D. E., “Self-induced back-action optical trapping in nanophotonic systems,” New Journal of Physics 17 (12), 123008 (2015).

### Example 2

### Assessment of the Protein-Protein Complex Detection Sensitivity of the SANE Sensor

### A. General

Peptide-presenting Major Histocompatibility Complex Class-I (pMHC) receptors being targeted by recombinant T-Cell Receptor-mimic (TCRm) antibodies can mediate the killing of specific cancer cells. High TCRm affinity combined with high avidity, meaning higher density binding to multiple ligand targets, can augment antitumor response significantly up to a limit set by autoimmunity. The cell copy number of pMHCs targeted by specific TCRms is an important determinant of avidity and therefore antitumor response. To advance the cancer immunotherapy field, technologies are needed to quantify both the number and heterogeneity of pMHC ligands in cells obtained from a patient tumor to select the antibodies with highest antitumor activity potential.

High-specificity tools that are needed to empirically assess whether a candidate TCRm antibody will target pMHCs in a patient's tumor are not readily available. Lower pMHC levels in cancer cells (10-100/cell) and expression heterogeneity in a tumor reduce the mean copy number. As a result, this limits the ability of commercial assays (enzyme-linked immunoassays (ELISAs), plasmon resonance techniques, antibody-dependent cellular cytotoxicity assays, complement dependent cytotoxicity assays, etc.) to detect many potential peptide targets. Since a needle biopsy collects >10⁶ cells, back of the envelope calculations show that commercial assays require cancer cell expansion in culture to acquire enough protein to detect a given pMHC target. However, cell culture induces clonal bias, where many possible targets are eliminated after several passes.

In this Example, this disclosure presents the results of a new AC nanopore sensing method that can be used to differentiate the specific binding of an antigen and antibody from non-specific binding at ultra-low analyte concentrations, down to low attomolar (aM). This work is helpful in eliminating the need for cancer cell expansion in testing tumor pMHC heterogeneity.

### B. METHODS AND RESULTS

Making pMHC's Commonly Overexpressed in GI, Breast, and Lung Cancers

In order to create pHMCs that are commonly overexpressed in gastrointestinal (GI), breast, and lung cancers, the recombinant peptides that are presented by HLA-A*0201 (HLA-A2) were synthesized, including cyclin-dependent kinase-2 (CdK-2; KIGEGTYGV), Systenin (SLMDHTIPEV), and TP53 (VVPCEPPEV).

### CdK-2 HLA-A2 Binding

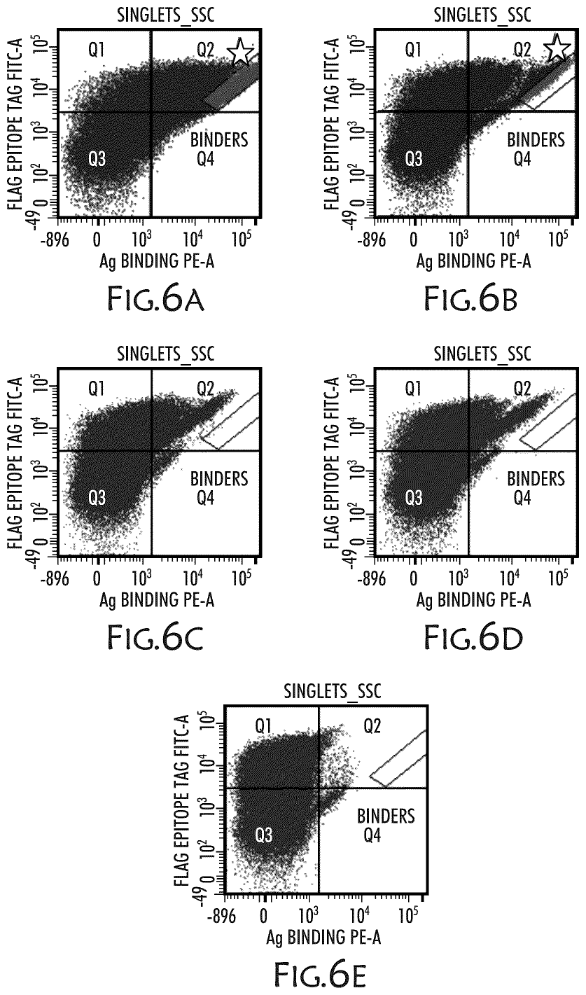

FACS sorting results for yeast expression is presented herein in FIG. 6. The results indicate that there is a subpopulation of yeast (gated region in histograms marked with a star in FIGS. 6A and 6B) that binds specifically to the CdK-2/HLA-A2 target at 10 nM (FIG. 6B) and 100 nM (FIG. 6A). However, this same subpopulation of yeast did not bind to negative control targets at a concentration of 100 nM (FIGS. 6C and 6D) or no antigen (FIG. 6E). The antibody expression on the surface of yeast was detected with an anti-FLAG tag-fluorescein isothiocyanate (FITC) conjugate (y-axis). Biotin-labeled pMHC antigen bound to yeast was detected with Streptavidin, R-Phycoerythrin (SA-PE) conjugate (x-axis).

### Assessment of the Protein Complex Detection Sensitivity of the SANE Sensor

The SANE sensor described in Example 1 was used herein. To test the sensitivity of the SANE sensor in detecting pMHCs and TCRms, titrations were performed for RAH, a pMHC peptide, and anti-RAH (TCRm) in homogeneous solutions as well as an equimolar heterogeneous solution of the mixture of the two (RAH-anti-RAH). Optical measurements, DC electrical measurements, and AC electrical measurements were taken.

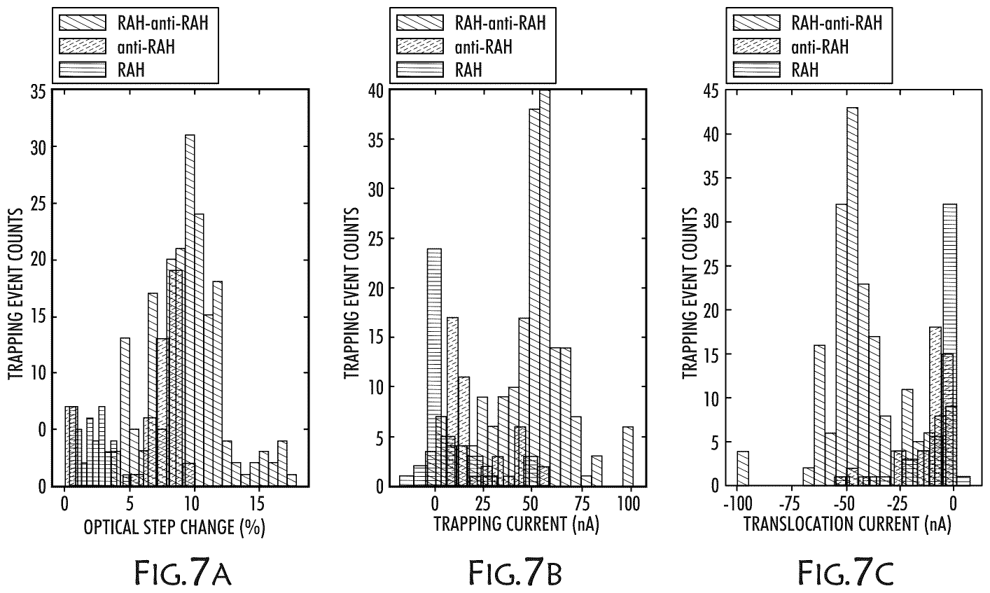

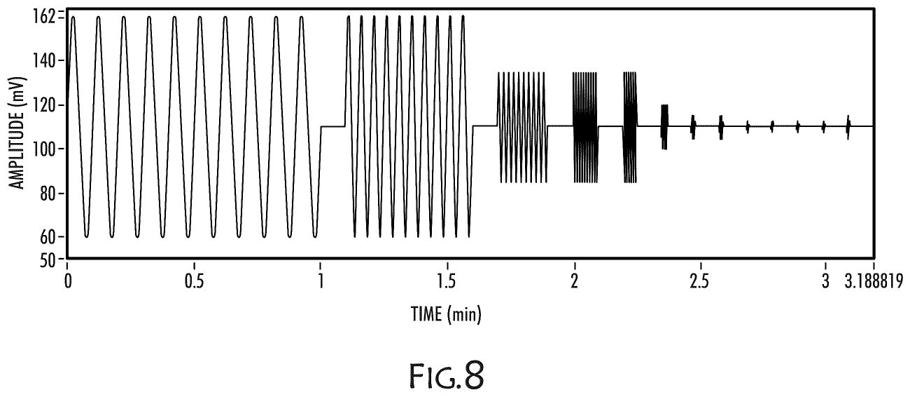

The equimolar solution mixture was initially incubated at 100 nM and then titrated down to concentrations ranging from 1 aM to 10 fM, and the trapping of pMHC-TCRms was observed at all concentrations. These dilutions are several orders of magnitude beyond the preliminary data of 0.1 pM. These new results show the extreme sensitivity of this plasmonic nanosensor technology. FIG. 7 shows the trapping event histograms for three parameters that can be used to identify trapped analytes: optical step change, trapping current, and translocation current. The diagonally hatched histograms (see legend of FIG. 7) represent the equimolar solution where trapping was observed for the RAH monomer, the anti-RAH antibody, and the bound complex. The outliers are likely representative of antibody aggregation. Horizontal hatching and middle hatching histograms (see legend of FIG. 7) represent measurements of the same three parameters in pure RAH and anti-RAH solutions, respectively. For the optical step change data, it was found that the optical step change increased with increasing mass of the trapped entity, and regarding the trapping current data, trapping current spikes are higher when the higher charge of the pMHC-TCRm complexes enter the optical trap. Regarding the translocation current, it was determined that nanopore translocation current spikes are the highest in the negative direction (current blockage) in the opposite direction when the trapped pMHC-TCRm complexes escape the optical trap of the plasmonic nanopore.

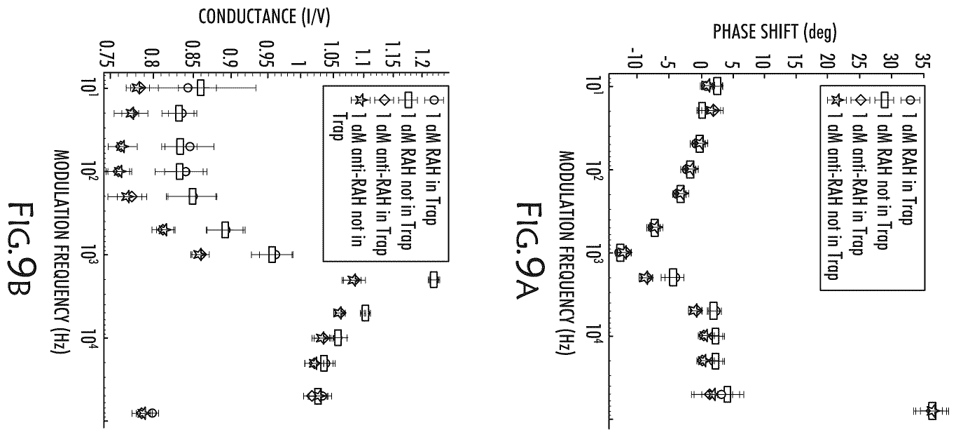

Subsequently, these DC optical-electrical data types were combined with the AC data types to generate multidimensional data sets for classifying and separating bound from unbound protein with higher accuracy. To expand the number of parameters used to identify trapped analytes, AC currents were introduced to the sensor to explore the frequency response of the sensor and the trapped proteins. A program was developed using Lab VIEW that delivers a pulse train that contains 10 cycles, each of 10, 20, 50, 100, 200, 500, 1000, 2000, 5000, 10000, 20000, 50000 and 100000 Hz (FIG. 8). This pulse train was used to drive the voltage clamp in the experimental setup. The driven command voltage results in a similar oscillation as current flow in the sensor. The phase and voltage/current relationship can be compared between the driving (command) voltage and the sensor response to test the feasibility of identifying differences in AC experimental data (phase delay and frequency-dependent conductance) as signatures to help further differentiate between different analytes trapped at the sensor.

FIG. 9A shows the phase shift plot (difference relative to the empty sensor), and FIG. 9B shows the conductance plot (ratio relative to the empty sensor) for each modulation frequency. All measurements were performed in 0.3 M KCl solution, both for the empty sensor and the solution with the analytes. From these plots, there is a clear separation between the analytes (RAH versus anti-RAH) for phase and conductance and at some frequencies. At approximately 1-5 kHz, there is maximum phase and gain difference between analytes. Interestingly, phase separation appears to be larger at higher modulation frequencies, whereas conductance separation between the two analytes is higher at lower frequencies. Notably, from the collected data, it did not appear that there was a significant separation between an analyte being inside the optical trap, or presumably just outside of it, at a concentration of 1 aM. There was indeed a larger separation at higher analyte concentrations (data not shown). Not intending to be bound by theory, from these observations, it was thought that the phase and gain are driven by the local solution volume in and around the plasmonic nanopore, not just by the single protein trapped near the mouth of the nanopore.

### Establishing Post-Decay Fits for RAH, Anti-RAH, and RAH-Anti-RAH Using the SANE Sensor

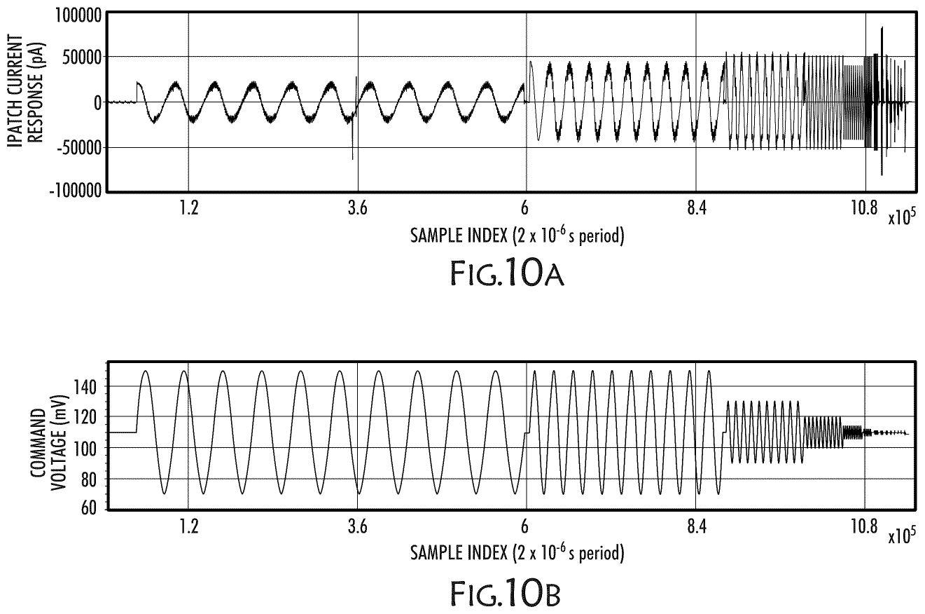

Using the method for the SANE sensor described in Example 1, AC bursts were applied to solutions of 1 aM RAH, 1 aM anti-RAH, and an equimolar heterogeneous solution of the mixture of the two (1 aM RAH-anti-RAH). An example plot of the Ipatch current response (pA, FIG. 10A) in response to the applied command voltage (mV, FIG. 10B) related to time(s) from an AC burst is shown in FIG. 10. The detected AC oscillations were caused by externally applied sinusoidal voltage bursts. These sinusoidal voltage bursts had frequencies spanning the 100 Hz to 100 kHz range logarithmically and were concatenated so that they were applied in immediate succession with a brief pause interval between them. More specifically, the following frequencies were used: 10, 20, 50, 100, 200, 500, 1000, 2000, 5000, 10000, 20000, 50000, 80000, and 100000 Hz. Each frequency ran for 10 cycles. The delay or pause between frequencies was set to 10 ms. However, this setting was in some cases modified by Lab VIEW because of signal buffer size. The amplitude was seen in the command voltage and was set to provide the maximum current response without saturating the filter.

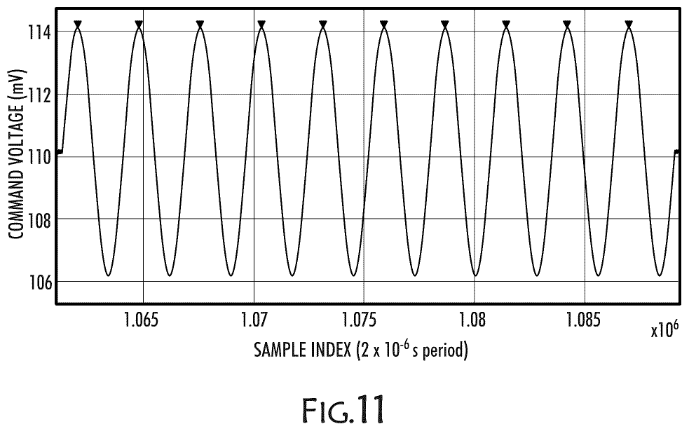

FIG. 11 show a close up of a section of one of the bursts of the command voltage plotted in FIG. 10B as related to time(s). The signal frequency plotted in the figure is indicative of only one of all frequencies used, as the same procedure was applied to all voltage bursts applied sequentially, as follows. A peak-finding algorithm was used to identify the locations of command voltage peaks along the time axis. The same peak-finding algorithm was applied to the corresponding response bursts detected by the Axopatch system. As both the applied command voltage and Axopatch response were recorded and digitized synchronously, the peak time difference in the sinusoidal command versus the sinusoidal response voltage defined a phase difference induced both by the analyte and the sensor system. Calibration measurements made before any experiments using analytes were performed and measured a similar phase difference for the empty sensor. Hence, when analyte measurements were performed, any net phase delay found in the signal beyond that of the empty sensor was attributed to the analyte. These analyte-induced phase differences at all frequencies used for the command voltage bursts yield AC-type features to subsequently help augment classification power for the analyte, in addition to the DC-type features obtained without the AC bursts.

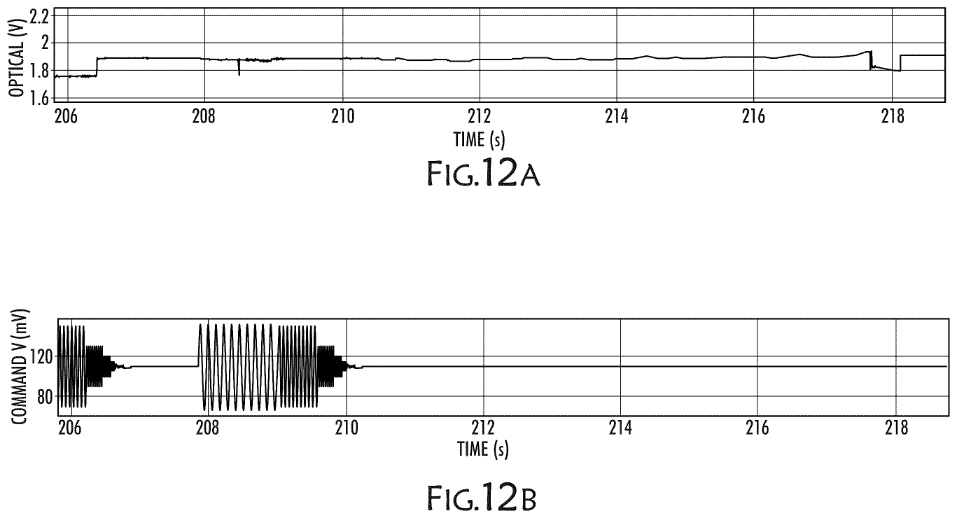

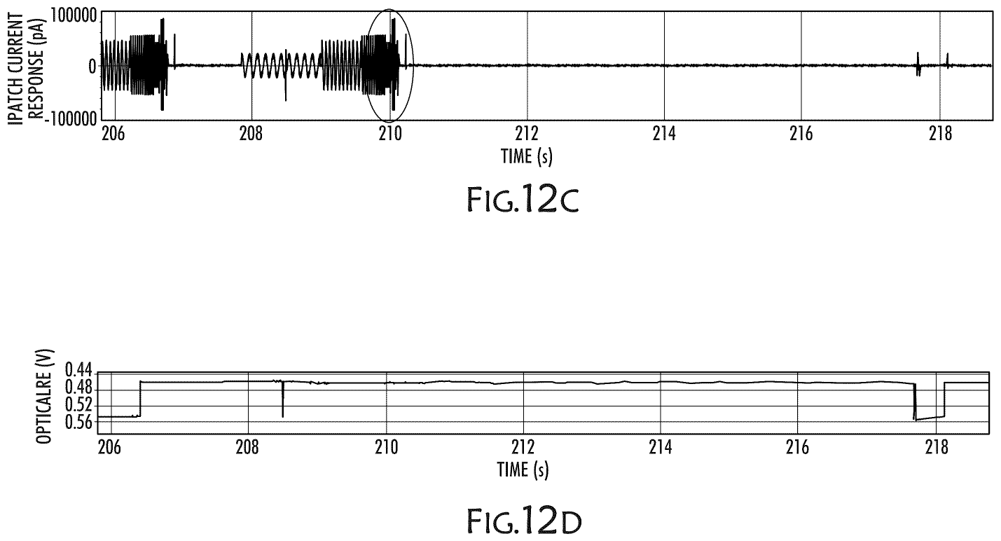

FIG. 12 shows another example plot of the optical current voltage (V, FIG. 12A), in response to the command voltage (mV, FIG. 12b), the Ipatch current response (pA, FIG. 12C), and the optical response (V, FIG. 12D) as related to time(s) for 1 aM RAH-anti-RAH. The sequence of frequency bursts is applied once, and the observed step increase indicates that a single RAH-anti-RAH complex is inside the optical trap of the SANE sensor.

FIG. 12C shows the combined analyte and sensor response for each frequency burst. The oval indicates that the response of the system at higher frequencies is higher in amplitude even though the amplitude of the applied bursts (FIG. 12C) was set to lower values at higher frequencies. Not intending to be bound by theory, it is believed this occurred because the senor chip becomes increasingly transparent to electrical signals at higher frequencies, so lower amplitude signals are applied to avoid detector saturation at those higher frequencies.

With reference once more to FIG. 12, one can detect a step-increase in the optical signal the lasts a little over 11 seconds (starting after 206 s and ending just before 218 s along the time axis). The duration of this step represents the trapping duration of the RAH-anti-RAH protein complex as the analyte in the optical trap of the SANE sensor. The start of a trapping event (the step-up after 206 s) was used as an electronic trigger to start the burst sequence with some delay to accommodate any electronic signal setting cause by a signal step change. Once the trapping event occurred, the burst sequence was applied as described above so that the AC voltage response of analyte and sensor are captured at different frequencies.

FIG. 12d in particular is related to a reference optical channel that measures back-scattered light from the SANE sensor in reflectance as opposed to transmittance. Measuring both in transmittance and reflectance helps validate that true trapping events occurred, as opposed to agglomerates that have a different forward and backward reflectance profiles.

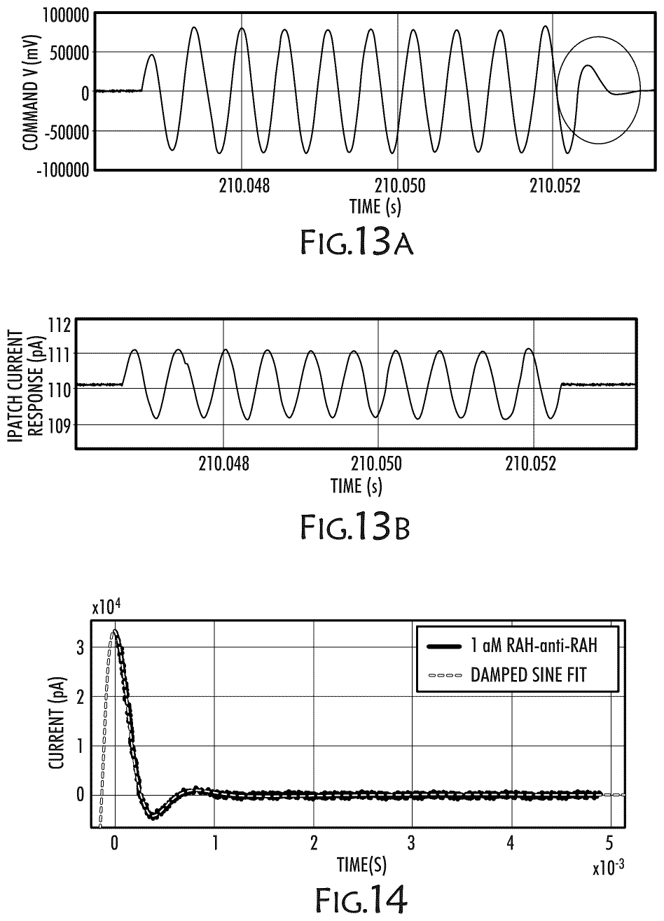

FIG. 13B is an expanded view of view of the sensor response when an analyte is trapped in it (an RAH-anti-RAH protein complex) in response to the externally applied command voltage shown in FIG. 13A. It is seen that in addition to a time delay in the response (phase delay of the sinusoidal wave relative to the command voltage), when the frequency burst is abruptly stopped (rightmost part of FIG. 13A) the sensor response continues briefly as a damped oscillation (oval in FIG. 13B). The shape of this damped oscillatory pattern is fit to a mathematical formula describing this behavior, and the fitting parameters of this formula (described further herein) provide additional metrics to help augment analyte classification. More specifically, for each AC burst, this post-drive decay was fit using Eq. 1 from Example 1 using a similar process described in Example 1. An example post-drive decay and its damped sine fit are shown in FIG. 14 for 1 aM RAH-anti-RAH.

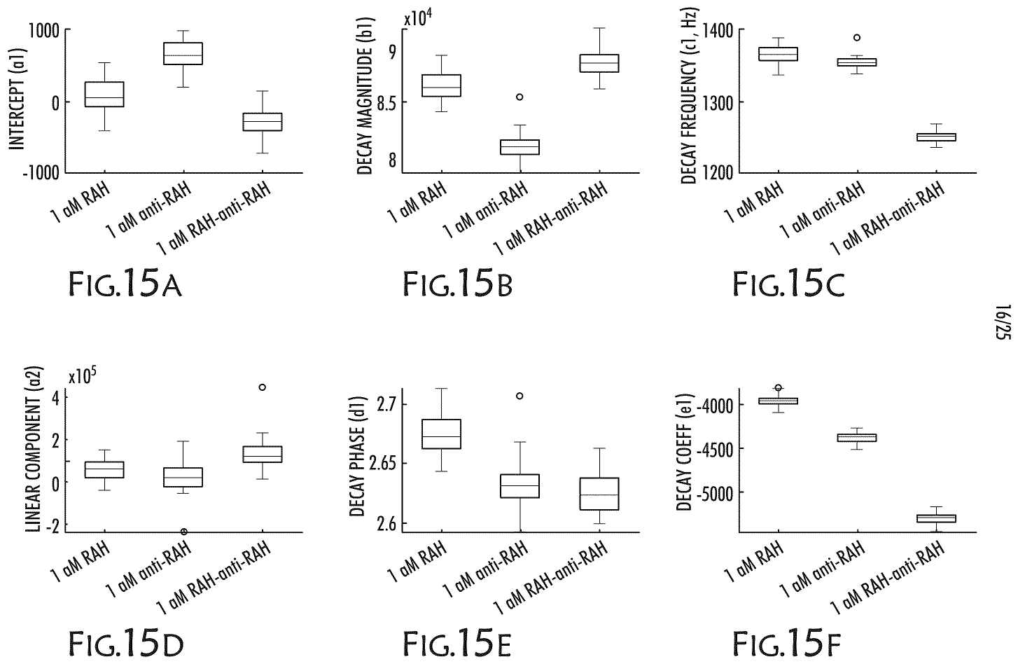

FIG. 15 is a box and whisker plot for the intercept of regression (a1), the magnitude of oscillation for regression (decay magnitude, b1), the frequency response of the post-drive decay (decay frequency, cl, Hz), the slope of the linear drift component (a2), the post-drive decay oscillation phase (decay phase, d1), and the decay coefficient for the damped oscillation envelope (e1) for 1 aM RAH, 1 aM anti-RAH, and 1 aM RAH-anti-RAH at 100 kHz driving frequency for the post-drive decay fits determined with Eq. 1. For the frequency of the post-drive decay in FIG. 15C (c1) and the decay coefficient for the damped oscillation envelope in FIG. 15F (e1), there was a clear decrease in these parameters when RAH and anti-RAH formed a complex at 1 aM at 100 kHz driving frequency. Thus, this method can distinguish the complex from the free components.

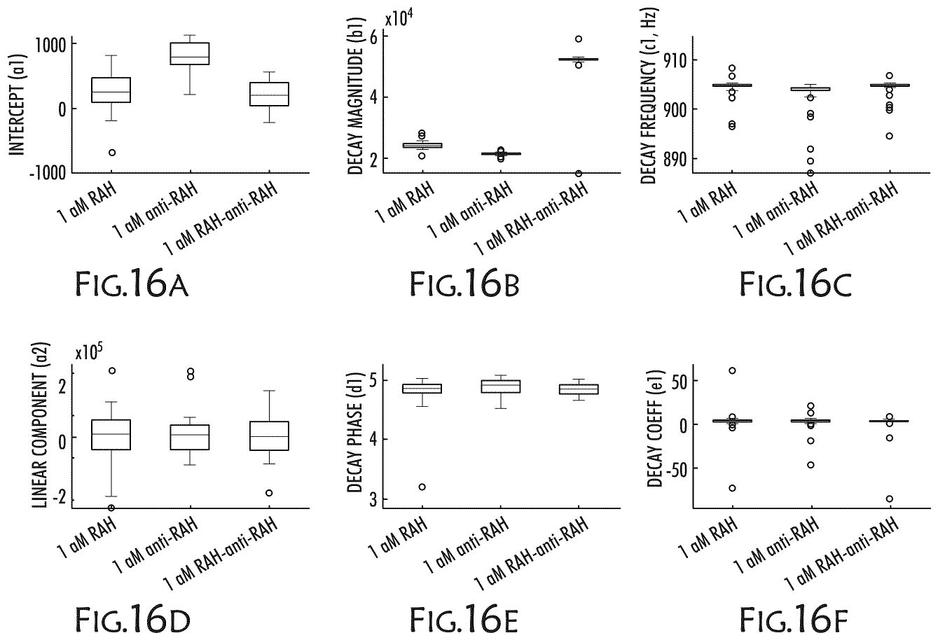

Further, FIG. 16 is a box and whisker plot for the intercept of regression (a1), the magnitude of oscillation for regression (decay magnitude, b1), the frequency response of the post-drive decay (decay frequency, cl, Hz), the slope of the linear drift component (a2), the post-drive decay oscillation phase (decay phase, d1), and the decay coefficient for the damped oscillation envelope (e1) for 1 aM RAH, 1 aM anti-RAH, and 1 aM RAH-anti-RAH at 1 KHz driving oscillation for the post-drive decay fits determined with Eq. 1. For the magnitude of oscillation for regression in FIG. 16b (b1), the binding of RAH and anti-RAH led to a clear increase in this parameter for 1 kHz driving oscillation. Again, there is a clear separation between RAH-anti-RAH and the free components, further supporting that this method can distinguish between the free components and bound complexes at ultra-low concentrations, especially when combining multiple features extracted from these model fits into a classification algorithm (as understood by one ordinary skill in the art, based upon the present disclosure).

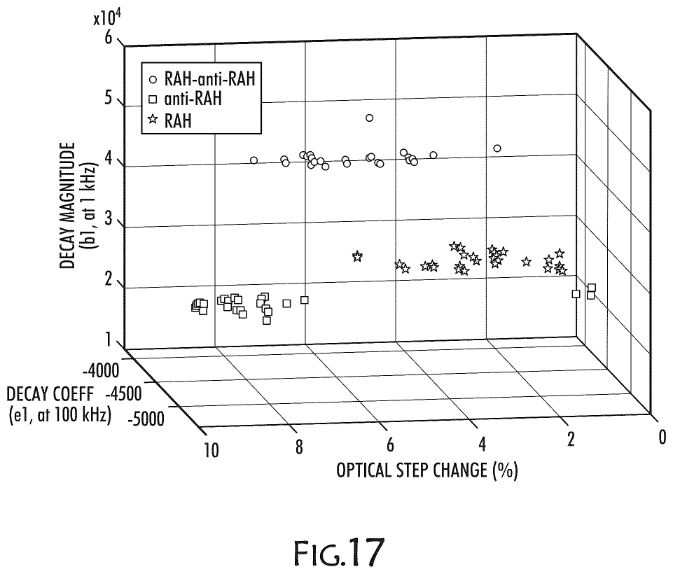

To further display the differences in these parameters upon binding, the data were plotted in a 3D multimodal display. FIG. 17 shows this 3D multimodal display of the optical step change (%) of 1 aM RAH, 1 aM anti-RAH, and 1 aM RAH-anti-RAH as related to the decay coefficient at 100 kHz driving oscillation and the decay magnitude at 1 KHz driving frequency as assessed from the post-drive decay fits. These data show a clear difference in the binding of RAH-anti-RAH and the free forms of RAH and anti-RAH Similarly, multiple features could be used by a classification algorithm to separate the data across a larger dimension set to enhance identification of analytes.

### C. CONCLUSION

This Example discloses results that show that the SANE sensor and AC method used with it are able to distinguish between bound and unbound antigen-antibody solutions at ultra-low analyte concentrations. Different dimensions, such as optical trapping time, decay coefficient, and decay magnitude, are able to differentiate between bound and unbound complexes. This will be useful for critical applications such as screening cancer patient tumors for pMHCs without the need for cell culture to generate enough protein material.

### Example 3

### Construction and Testing of an ITP Platform to Separate Protein Complexes from Ligands

This Example describes an integrated isotachophoresis (ITP) platform that was developed to mount on top of the nanosensor described in Example 1 to concurrently increase the concentration of TCRm antibodies and target pMHCs while separating them from unbound proteins of different sizes and charge. For ˜104 cancer cells per assay, each cell expressing 10-100 copies of a specific pMHC, 1-10 nM of targeted complexes will exist in the concentrated analyte plug.

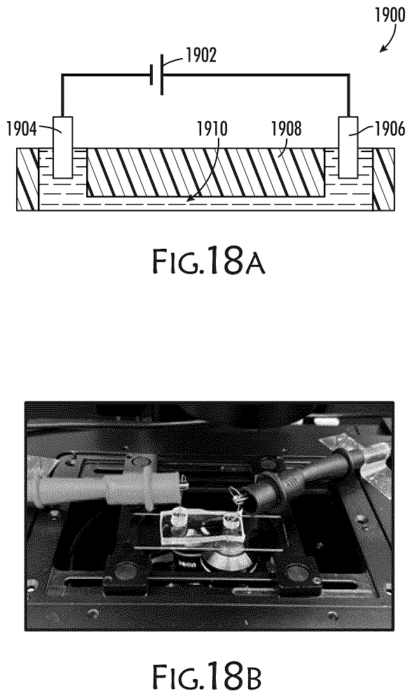

FIG. 18A is a schematic of the system. The overall design comprises a channel structure with a diverging section starting from 1 mm from the cathode to anode reservoir. Pt electrodes are submerged into terminating electrolyte (TE) and leading electrolyte (LE) reservoirs to supply constant voltage bias through the channel. During testing, the loaded microchip is fixed onto the stage of a confocal microscope for the observation of separation dynamics, which is shown in FIG. 18b. Chips are formed from PDMS because of its low cost, ease of fabrication, and optical clarity.

FIG. 19A-F is an illustration of the steps to fabricate the PDMS channel, showing the wafer layer (FIG. 19A), the photoresist layer (FIG. 19b), and the UV light penetrating around the photomask (FIG. 19c), ultimately leading the mold shown in FIG. 19D. FIG. 19E illustrates the replica molding process, and FIG. 19G shows the final channel structure. FIG. 19H is a schematic of the channel pattern with dimensions.

Before testing, to decontaminate the channel, the chip is cleaned with a 15% bleach solution, rinsed with deionized water, and vacuumed inside to remove any bleach residue. The channel is then flushed with LE solution several times before the TE reservoir is rinsed with deionized water to strongly dilute any LE solution residue before filling the reservoir with TE solution.

Because of the scarcity of the target protein samples (pMHC ligands and TCRms), low-cost samples comprising anionic dye-labeled Dextran conjugates weighing 40 kDa and 70 kDa were used to test the functionality of the experimental setup. Samples were diluted down to 0.25 ng/ml in TE solution before injection. Two sets of circular PDMS extensions were attached on top of the channels using plasma bonding to have better control over the volume added to the reservoirs and to help keep the electrodes stationary. Two sets of Pt electrodes were connected to the power supply (Sorensen XHR600-1.7 DC) and submerged into the cathode and anode reservoirs filled with TE and LE solutions, respectively. The loaded microchip was mounted above a 4× objective lens of an Olympus Confocal Microscope FV 3000. Constant voltage up to 600 V was applied by the power supply. TE solution containing HEPES and LE solution containing HCl were titrated to the same pH with Tris. HEPES was chosen because of its low electrophoretic mobility, and the chloride ion was selected for its high electrophoretic mobility. Tris was the counterion. The LE and TE solutions also contained 1% (w/v) polyvinylpyrrolidone to suppress the effect of electroosmotic flow.

Additional non-limiting, example embodiments are further described below.

Embodiment 1. A method of sensing comprising:

(1) providing a sensor comprising (a) a first layer having at least one single nanohole structure or at least one dual nanohole structure, and (b) a second layer having at least one nanopore, wherein the single nanohole structure comprises only one nanohole, wherein the dual nanohole structure comprises a first nanohole and a second nanohole connected by a gap, and wherein the one nanohole or the gap of the first layer is aligned with the nanopore of the second layer in a direction corresponding to a translocation direction across the first and second layers;
(2) providing a test sample comprising an analyte;
(3) contacting the test sample with the first layer of the sensor;
(4) irradiating the single nanohole structure or the dual nanohole structure of the first layer of the sensor with a beam of electromagnetic radiation;
(5) optically trapping the analyte in the single nanohole structure or in the dual nanohole structure and/or in the gap of the first layer of the sensor;
(6) applying a first electric field across the nanopore to draw one or more of the analytes into the nanopore, wherein the first electric field comprises a direct current (DC) electric field;
(7) applying a second electric field across the nanopore after applying the first electric field, wherein the second electric field comprises a pulsed, modulated, or alternating current (AC) electric field; and
(8) measuring one or more of: (a) change in current and/or phase across the nanopore during application of the second electric field while the analyte is optically trapped and/or during one or more translocation events of the analyte through the nanopore; or (b) at least one kinetic parameter of the analyte within the nanopore after removing or turning off the second electric field.

Embodiment 2. The method of Embodiment 1, wherein the at least one kinetic parameter is measured while the analyte decelerates or comes to a stop while optically trapped.

Embodiment 3. The method of Embodiment 1 or Embodiment 2, wherein the at least one kinetic parameter comprises one or more of the following: equilibrium dissociation constant (K_{d}), binding on-rate (kₒₙ), binding off-rate (k_{off}), bound fraction (i.e., the fraction of analyte that is in a bound or complexed state rather than an unbound or uncomplexed state), analyte size (volume), analyte charge (effective charge on the outer surface of the analyte), and analyte conformation.

Embodiment 4. The method of any of the preceding Embodiments, wherein measuring change in current and/or phase further comprises determining a charge of a translocating analyte.

Embodiment 5. The method of any of the preceding Embodiments, wherein measuring change in current and/or phase further comprises determining a dielectric constant of a translocating analyte.

Embodiment 6. The method of any of the preceding Embodiments further comprising measuring a surface plasmon resonance of the single nanohole structure or the dual nanohole structure after optically trapping the analyte in the single nanohole structure or in the dual nanohole structure and/or in the gap of the first layer of the sensor.

Embodiment 7. The method of Embodiment 6, wherein measuring the surface plasmon resonance further comprises determining the mass of an optically trapped analyte.

Embodiment 8. The method of any of the preceding Embodiments, wherein the analyte comprises complexed and/or non-complexed biomolecules.

Embodiment 9. The method of any of the preceding Embodiments, wherein the test sample is a biological sample obtained from an animal or human subject, such as a human patient or animal patient in need of diagnosis.

Embodiment 10. The method of Embodiment 9, wherein the analyte comprises a pMHC or pMHC component.

Embodiment 11. The method of Embodiment 9, wherein the analyte comprises a HLA-A2 pHMC or HLA-A2 pMHC component.

Embodiment 12. The method of Embodiment 9, wherein the analyte comprises a TCRm antibody.

Embodiment 13. The method of Embodiment 9, where in the analyte comprises a TCRm antibody against a HLA-A2 pHMC or against a HLA-A2 pHMC component.

Embodiment 14. The method of any of Embodiments 1-7, wherein the analyte comprises an inorganic nanoparticle.

Embodiment 15. The method any of the preceding Embodiments, wherein the test sample is concentrated prior to contacting the test sample with the first layer of the sensor.

Embodiment 16. The method of Embodiment 15, wherein the test sample is concentrated using isotachophoresis (ITP).

Embodiment 17. The method of Embodiment 15, wherein the test sample is concentrated using an ITP microchannel structure.

Embodiment 18. The method of Embodiment 17, wherein the ITP microchannel structure is disposed over the first layer of the sensor.

Embodiment 19. The method of Embodiment 18, wherein the ITP microchannel structure forms a unitary chip with the first layer and the second layer of the sensor.

Various embodiments of the present invention have been described in fulfillment of the various objectives of the invention. It should be recognized that these embodiments are merely illustrative of the principles of the present invention. Numerous modifications and adaptations thereof will be readily apparent to those skilled in the art without departing from the spirit and scope of the invention.

1. A method of sensing comprising:
providing a sensor comprising:
a first layer having at least one single nanohole structure or at least one dual nanohole structure, and
a second layer having at least one nanopore,
wherein the single nanohole structure comprises only one nanohole,
wherein the dual nanohole structure comprises a first nanohole and a second nanohole connected by a gap, and
wherein the one nanohole or the gap of the first layer is aligned with the nanopore of the second layer in a direction corresponding to a translocation direction across the first and second layers;
providing a test sample comprising an analyte;
contacting the test sample with the first layer of the sensor;
irradiating the single nanohole structure or the dual nanohole structure of the first layer of the sensor with a beam of electromagnetic radiation;
optically trapping the analyte in the single nanohole structure or in the dual nanohole structure and/or in the gap of the first layer of the sensor;
applying a first electric field across the nanopore to draw one or more of the analytes into the nanopore, wherein the first electric field comprises a direct current (DC) electric field;
applying a second electric field across the nanopore after applying the first electric field, wherein the second electric field comprises a pulsed, modulated, or alternating current (AC) electric field; and
measuring one or more of:
change in current and/or phase across the nanopore during application of the second electric field while the analyte is optically trapped and/or during one or more translocation events of the analyte through the nanopore; or
at least one kinetic parameter of the analyte within the nanopore after removing or turning off the second electric field.

2. The method of claim 1, wherein the at least one kinetic parameter is measured while the analyte decelerates or comes to a stop while optically trapped.

3. The method of claim 1, wherein the at least one kinetic parameter comprises one or more of the following: equilibrium dissociation constant (K_{d}), binding on-rate (kₒₙ), binding off-rate (k_{off}), and bound fraction.

4. The method of claim 1, wherein measuring change in current and/or phase further comprises determining a charge of a translocating analyte.

5. The method of claim 1, wherein measuring change in current and/or phase further comprises determining a dielectric constant of a translocating analyte.

6. The method of claim 1 further comprising:
measuring a surface plasmon resonance of the single nanohole structure or the dual nanohole structure after optically trapping the analyte in the single nanohole structure or in the dual nanohole structure and/or in the gap of the first layer of the sensor.

7. The method of claim 6, wherein measuring the surface plasmon resonance further comprises determining the mass of an optically trapped analyte.

8. The method of claim 1, wherein the analyte comprises complexed and/or non-complexed biomolecules.

9. The method of claim 1, wherein the test sample is a biological sample obtained from an animal or human subject.

10. The method of claim 9, wherein the analyte comprises a pMHC or pMHC component.

11. The method of claim 9, wherein the analyte comprises a HLA-A2 pHMC or HLA-A2 pMHC component.

12. The method of claim 9, wherein the analyte comprises a TCRm antibody.

13. The method of claim 9, where in the analyte comprises a TCRm antibody against a HLA-A2 pHMC or against a HLA-A2 pHMC component.

14. The method of claim 1, wherein the analyte comprises an inorganic nanoparticle.

15. The method of claim 1, wherein the test sample is concentrated prior to contacting the test sample with the first layer of the sensor.

16. The method of claim 15, wherein the test sample is concentrated using isotachophoresis (ITP).

17. The method of claim 15, wherein the test sample is concentrated using an ITP microchannel structure.

18. The method of claim 17, wherein the ITP microchannel structure is disposed over the first layer of the sensor.

19. The method of claim 18, wherein the ITP microchannel structure forms a unitary chip with the first layer and the second layer of the sensor.
